(19) United States
(12) Patent Application Publication
(10) Pub. No.: US 2013/0281445 A1
(43) Pub. Date: Oct. 24, 2013

(54) COMPOUNDS USEFUL AS INHIBITORS OF CHOLINE KINASE

(71) Applicants: Simon Everitt, Beaconsfield (GB); Ronald Knegtel, Abingdon (GB); Michael Mortimore, Burford (GB); Alistair Rutherford, Abingdon (GB)

(72) Inventors: Simon Everitt, Beaconsfield (GB); Ronald Knegtel, Abingdon (GB); Michael Mortimore, Burford (GB); Alistair Rutherford, Abingdon (GB)

(21) Appl. No.: 13/623,962

(22) Filed: Sep. 21, 2012

Related U.S. Application Data
(60) Provisional application No. 61/537,916, filed on Sep. 22, 2011.

(51) Int. Cl. C07D 453/02; A61K 45/06; A61K 31/498; A61K 31/439; A61K 31/538

(52) U.S. Cl. CPC C07D 453/02; A61K 31/439; A61K 31/538; A61K 31/498; A61K 45/06
USPC 514/230.5; 546/133; 514/305; 544/105; 544/353; 514/249; 435/184

(57) ABSTRACT

The present invention relates to compounds useful as inhibitors of choline kinase. The invention also provides pharmaceutically acceptable compositions comprising said compounds and methods of using the compositions in the treatment of various disease, conditions, or disorders. The invention also provides processes for preparing compounds of the inventions.

### CROSS REFERENCE TO RELATED APPLICATIONS

This present invention claims the benefit, under 35 U.S.C. §119, of U.S. Provisional Application No. 61/537,916, filed Sep. 22, 2011, the entire contents of each of the above applications being incorporated herein by reference.

### TECHNICAL FIELD OF THE INVENTION

The present invention relates to compounds useful as inhibitors of choline kinase. The invention also provides pharmaceutically acceptable compositions comprising the compounds of the invention. The invention provides methods of treating various diseases, disorders, and conditions using the compounds of the invention. The invention also provides processes for preparing the compounds of the invention.

### BACKGROUND OF THE INVENTION

Choline Kinase (ChoK) is a cytosolic enzyme that catalyses the Mg.ATP-dependent phosphorylation of choline as the first step in the Kennedy pathway, in which choline is incorporated into phosphatidylcholine (PtdCho) (Kennedy, 1957 Annual Review of Biochemistry, 26, 119-48). In this reaction, choline is first converted into phosphocholine (PCho), which then reacts with CTP to form CDP-choline. The PCho moiety is then transferred to diacylglycerol to produce PtdCho. This pathway is the major source of PtdCho, which is a highly abundant class of phospholipids in mammalian cellular membranes (Gibellini & Smith, 2010; Life, 63, 414-428).

In mammals the Choline Kinase family of proteins is comprised of two isoforms, Choline Kinase alpha (ChoKα) and Choline Kinase beta (ChoKβ) (Aoyama et al, 2004. Progress in Lipid Research, 43, 266-281). ChoKα has been identified as an oncogene that mediates human cell transformation and induces in vivo tumorigenesis (Ramirez de Molina et al, 2005. Cancer Research, 65, 5647-5653), and forced over-expression has been shown to cause increased tumor formation and aggressiveness of disease (Hernando et al, 2009. Oncogene, 28, 2425-2435). In addition, over-expression of ChoKα increases invasiveness and drug resistance to 5-fluorouracil of human breast cancer cells (Shah et al, 2010. NMR in Biomedicine, 23: 633-642). The increase in ChoK activity results in elevated levels of PCho, a putative second messenger involved in proliferation (Cuadrado et al, 1993. Oncogene, 8, 2959-2968).

ChoKα has been implicated in the carcinogenic process, since several groups have reported increased ChoKα expression and increased ChoKα activity in several different types of clinical tumors (including lung, colon, breast, prostate, bladder, ovarian), as well as in different human cancer cell lines (Nakagami et al, 1999. Japanese Journal of Cancer Research 90, 419-424; Ramirez de Molina et al, 2002. Biochemical and Biophysical Research Communications, 296, 580-583; Iorio et al, 2005. Cancer Research, 65, 9369-9376; Gabellieri et al, 2009. NMR in Biomedicine, 22, 456-461; Hernando et al, 2009. Oncogene, 28, 2425-2435). High expression of ChoKα has also been associated with poor clinical outcome and high histological tumor grade (Ramirez de Molina et al, 2007. Lancet Oncology, 8, 889-897; Ramirez de Molina et al, 2002. Oncogene, 21, 4317-4322). For this reason it has been proposed to use ChoKα as a prognostic marker for cancer progression as well as a molecular target for the development of novel cancer therapeutic agents (Glunde et al, 2006. Expert Reviews of Molecular Diagnostics, 6, 821-829)

The proposed mode of action in cancer cells is that ChoKα inhibition results in a reduction in PCho levels, which culminates in defects in both PtdCho and sphingomyelin (SM) synthesis. This results in cell death through a reduction in survival signaling and an increase in apoptosis due to an increase in the intracellular levels of ceramide, and a decrease in signaling through the MAPK and PI3K/AKT pathways (Rodriguez-Gonzalez et al, 2004. Oncogene, 23, 8247-8259; Yalcin et al, 2009. Oncogene, 29, 139-149). In contrast, ChoKα inhibition in non-cancer cells has been shown to cause a reversible cell cycle arrest (Rodriguez-Gonzalez et al, 2004. Oncogene, 23, 8247-8259; Rodriguez-Gonzalez et al, 2005. International Journal of Oncology, 26, 999-1008). As such, due to the relevance of ChoKα in human carcinogenesis, ChoKα inhibition constitutes an efficient anti-tumor strategy.

The use of small interfering RNA (siRNA) or small hairpin RNA plasmids (shRNA) of ChoKα has been shown to reduce intracellular PCho levels and reduce viability of different cancer cell lines in vitro, without affecting normal primary cells (Mori et al, 2007. Cancer Research, 67, 11284-11290; Banez-Coronel et al, 2008. Current Cancer Drug Targets, 8, 709-719; Yalcin et al, 2009. Oncogene, 29, 139-149), and when used in vivo, ChoKα depletion has been shown to result in a reduction of tumor growth (Banez-Coronel et al, 2008. Current Cancer Drug Targets, 8, 709-719; Krishnamachary et al, 2009. Cancer Research, 69, 3464-3471). In addition, it was demonstrated that ChoKα down-regulation using siRNA increases the anti-cancer effect of 5-fluorouracil in breast cancer cells (Mori et al, 2007. Cancer Research, 67, 11284-11290).

In an effort to develop new anti-cancer therapies, numerous compounds have been synthesized and reported as ChoKα inhibitors, most of which are derivatives of hemicholinium-3, a known inhibitor of ChoKα with a structural homology to choline (Cannon, 1994. Medicinal Research Reviews, 14, 505-531; Hernandez-Alcoceba et al, 1997. Oncogene, 15, 2289-2301; Lacal, 2001. IDrugs, 4, 419-426). It has been found that pharmacological inhibition of ChoKα in different cancer cell types resulted in growth arrest and apoptosis, with minimal effect on non-cancer cells (Rodriguez-Gonzalez et al, 2004. Oncogene, 23, 8247-8259; Rodriguez-Gonzalez et al, 2005. International Journal of Oncology, 26, 999-1008; Ramirez de Molina et al, 2007. Lancet Oncology, 8, 889-897; Hernando et al, 2009. Oncogene, 28, 2425-2435). In addition, ChoKα inhibitors have also proven to be potent antitumor drugs in vivo (Hernandez-Alcoceba et al, 1999. Cancer Research, 59, 3112-3118; Ramirez de Molina et al, 2004. Cancer Research, 64, 6732-6739; Hernando et al, 2009. Oncogene, 28, 2425-2435).

Choline Kinase is also the first enzyme in the Kennedy pathway (CDP-choline pathway) for the biosynthesis of the most essential phospholipid, phosphatidylcholine, in malaria-causing Plasmodium parasites. Based on pharmacological and genetic data, the de novo biosynthesis of PtdCho appears to be essential for the intraerythrocytic growth and survival of the malaria parasite. An inhibitor of Plasmodium Falciparum Choline Kinase, hexadecyltrimethylammonium bromide, showed very potent antimalarial activity in vitro against the Plasmodium falciparum parasite by causing a decrease in phosphocholine, which in turn causes a decrease in phosphatidylcholine biosynthesis, resulting in death of the parasite. This highlights the potential for ChoK inhibitors in the fight against malaria (Choubey et al, 2006. Biochimica et Biophysica Acta, 1760, 1027-38; Choubey et al, 2007. Antimicrobial Agents and Chemotherapy, 51, 696-706; Alberge et al, 2009. Biochemical Journal, 425, 149-58; Dechamps et al, 2010. Molecular and Biochemical Parasitology, 173, 69-80).

Accordingly, there is a need for the development of choline inhibitors for the treatment of the various diseases listed above.

### SUMMARY OF THE INVENTION

This invention relates to compounds and compositions useful as kinase inhibitors. Compounds of this invention, and pharmaceutically acceptable compositions thereof, are effective as inhibitors of kinases. In some embodiments, these compounds are effective as inhibitors of choline kinase. These compounds have the formula I, as defined herein, or a pharmaceutically acceptable salt thereof.

These compounds and pharmaceutically acceptable compositions thereof are useful for treating or preventing a variety of diseases, disorders or conditions, including, but not limited to cancer and malaria. These compounds are also useful for the study of kinases in biological and pathological phenomena; the study of intracellular signal transduction pathways mediated by such kinases; and the comparative evaluation of new kinase inhibitors.

This invention also provides processes for making the compounds of this invention.

### DETAILED DESCRIPTION OF THE INVENTION

This invention describes compounds of Formula I:

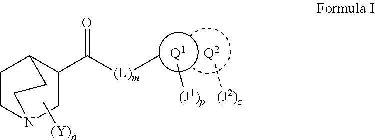

wherein:
Y is bonded to any carbon atom of the quinuclidine ring and is independently C₁₋₃ aliphatic, —CF₃, —CN, halo, ═O, —OH, —O(C₁₋₃aliphatic), NH₂, or NH(C₁₋₃ aliphatic);
   n is 0-4;
L is a C₁₋₂ alkyl;
   m is 0 or 1;
Q¹ is a 5 or 6 membered aromatic or non-aromatic ring having 0-2 heteroatoms independently selected from nitrogen, oxygen, or sulfur; wherein Q¹ is optionally substituted with p occurrences of J¹ and is optionally fused with Q²;
Q² is a 5 or 6 membered aromatic or non-aromatic ring having 0-2 heteroatoms independently selected from nitrogen, oxygen, or sulfur, wherein Q² is optionally substituted with z occurrences of J²;
J¹ is —Cl, —F, —Br, —NR²R³, —OCF₃, —O(C₁₋₄ aliphatic), -methyl, -ethyl, -tert-butyl, -propyl, —CF₃, —CN, or phenyl, wherein said J¹ is independently and optionally substituted with 1-3 occurrences of halo, —O(C₁₋₄aliphatic), —CN, or —OH;
R² is H or C₁₋₆ alkyl;
R³ is H or C₁₋₆ alkyl;
or R² and R³, taken together with the atom to which they are bound, form a 4-8 membered heterocyclic ring having 1-2 heteroatoms selected from oxygen, nitrogen, or sulfur;
p is 0, 1, 2, or 3, wherein p is not 0 when m is 0, and p is at least 2 when Q¹ is a phenyl, J¹ is Cl or methyl, and Q² is absent;
J² is C₁₋₃ alkyl, halo, or CF₃; and
z is 0, 1, 2, or 3.

Compounds of this invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As described herein, a specified number range of atoms includes any integer therein. For example, a group having from 1-4 atoms could have 1, 2, 3, or 4 atoms.

As described herein, compounds of the invention may optionally be substituted with one or more substituents, such as are illustrated generally herein, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

The term “stable”, as used herein, refers to compounds that are not substantially altered when subjected to conditions to allow for their production, detection, recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “aliphatic” or “aliphatic group”, as used herein, means a straight-chain (i.e., unbranched), branched, or cyclic, substituted or unsubstituted hydrocarbon chain that is completely saturated or that contains one or more units of unsaturation that has a single point of attachment to the rest of the molecule.

Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms, and in yet other embodiments aliphatic groups contain 1-4 aliphatic carbon atoms. Aliphatic groups may be linear or branched, substituted or unsubstituted alkyl, alkenyl, or alkynyl groups. Specific examples include, but are not limited to, methyl, ethyl, isopropyl, n-propyl, sec-butyl, vinyl, n-butenyl, ethynyl, and tert-butyl.

The term “cycloaliphatic” (or “carbocycle” or “carbocyclyl”) refers to a monocyclic C3-C8 hydrocarbon or bicyclic C8-C12 hydrocarbon that is completely saturated or that contains one or more units of unsaturation, but which is not aromatic, that has a single point of attachment to the rest of the molecule wherein any individual ring in said bicyclic ring system has 3-7 members. Examples of cycloaliphatic groups include, but are not limited to, cycloalkyl and cycloalkenyl groups. Specific examples include, but are not limited to, cyclohexyl, cyclopropenyl, and cyclobutyl.

The term “heterocycle”, “heterocyclyl”, or “heterocyclic” as used herein means non-aromatic, monocyclic, bicyclic, or tricyclic ring systems in which one or more ring members are an independently selected heteroatom. In some embodiments, the “heterocycle”, “heterocyclyl”, or “heterocyclic” group has three to fourteen ring members in which one or more ring members is a heteroatom independently selected from oxygen, sulfur, nitrogen, or phosphorus, and each ring in the system contains 3 to 7 ring members.

Examples of heterocycles include, but are not limited to, 3-1H-benzimidazol-2-one, 3-(1-alkyl)-benzimidazol-2-one, 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolane, benzodithiane, and 1,3-dihydro-imidazol-2-one.

Cyclic groups, (e.g. cycloaliphatic and heterocycles), can be linearly fused, bridged, or spirocyclic.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation. As would be known by one of skill in the art, unsaturated groups can be partially saturated or fully unsaturated. Examples of partially unsaturated groups include, but are not limited to, butene, cyclohexene, and tetrahydropyridine. Examples of fully unsaturated groups include, but are not limited to, phenyl, cyclooctatetraene, pyridyl, and thienyl.

The term “alkoxy”, or “thioalkyl”, as used herein, refers to an alkyl group, as previously defined, attached through an oxygen (“alkoxy”) or sulfur (“thioalkyl”) atom.

The terms “haloalkyl”, “haloalkenyl”, “haloaliphatic”, and “haloalkoxy” mean alkyl, alkenyl or alkoxy, as the case may be, substituted with one or more halogen atoms. This term includes perfluorinated alkyl groups, such as —CF₃ and —CF₂CF₃.

The terms “halogen”, “halo”, and “hal” mean F, Cl, Br, or I.

The term “aryl” used alone or as part of a larger moiety as in “aralkyl”, “aralkoxy”, or “aryloxyalkyl”, refers to monocyclic, bicyclic, and tricyclic ring systems having a total of five to fourteen ring members, wherein at least one ring in the system is aromatic and wherein each ring in the system contains 3 to 7 ring members. The term “aryl” may be used interchangeably with the term “aryl ring”.

The term “heteroaryl”, used alone or as part of a larger moiety as in “heteroaralkyl” or “heteroarylalkoxy”, refers to monocyclic, bicyclic, and tricyclic ring systems having a total of five to fourteen ring members, wherein at least one ring in the system is aromatic, at least one ring in the system contains one or more heteroatoms, and wherein each ring in the system contains 3 to 7 ring members. The term “heteroaryl” may be used interchangeably with the term “heteroaryl ring” or the term “heteroaromatic”. Examples of heteroaryl rings include, but are not limited to, 2-furanyl, 3-furanyl, N-imidazolyl, 2-imidazolyl, 4-imidazolyl, 5-imidazolyl, benzimidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, benzofuryl, benzothiophenyl, indolyl (e.g., 2-indolyl), pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

The term “protecting group” and “protective group” as used herein, are interchangeable and refer to an agent used to temporarily block one or more desired functional groups in a compound with multiple reactive sites. In certain embodiments, a protecting group has one or more, or preferably all, of the following characteristics: a) is added selectively to a functional group in good yield to give a protected substrate that is b) stable to reactions occurring at one or more of the other reactive sites; and c) is selectively removable in good yield by reagents that do not attack the regenerated, deprotected functional group. As would be understood by one skilled in the art, in some cases, the reagents do not attack other reactive groups in the compound. In other cases, the reagents may also react with other reactive groups in the compound. Examples of protecting groups are detailed in Greene, T. W., Wuts, P. G in “Protective Groups in Organic Synthesis”, Third Edition, John Wiley & Sons, New York: 1999 (and other editions of the book), the entire contents of which are hereby incorporated by reference. The term “nitrogen protecting group”, as used herein, refers to an agent used to temporarily block one or more desired nitrogen reactive sites in a multifunctional compound. Preferred nitrogen protecting groups also possess the characteristics exemplified for a protecting group above, and certain exemplary nitrogen protecting groups are also detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

In some embodiments, a methylene unit of an alkyl or aliphatic chain is optionally replaced with another atom or group. Examples of such atoms or groups include, but are not limited to, —NR²—, —O—, —C(O)—, —C(═N—CN)—, —C(═NR²)—, —C(═NOR²)—, —S—, —SO—, and —SO₂—. These atoms or groups can be combined to form larger groups. Examples of such larger groups include, but are not limited to, —OC(O)—, —C(O)CO—, —CO₂—, —C(O)NR²—, —C(═N—CN), —NR²CO—, —NR²C(O)O—, —SO₂NR²—, —NR²SO₂—, —NR²C(O)NR²—, —OC(O)NR²—, and —NRSO₂NR²—, wherein R² is defined herein.

Unless otherwise indicated, the optional replacements form a chemically stable compound. Optional replacements can occur both within the chain and/or at either end of the chain; i.e. both at the point of attachment and/or also at the terminal end. Two optional replacements can also be adjacent to each other within a chain so long as it results in a chemically stable compound. The optional replacements can also completely replace all of the carbon atoms in a chain. For example, a C₃ aliphatic can be optionally replaced by —NR²—, —C(O)—, and —NR²— to form —NR²C(O)NR²— (a urea).

Unless otherwise indicated, if the replacement occurs at the terminal end, the replacement atom is bound to an H on the terminal end. For example, if a methylene unit of —CH₂CH₂CH₃ were optionally replaced with —O—, the resulting compound could be —OCH₂CH₃, —CH₂OCH₃, or —CH₂CH₂OH.

Unless otherwise indicated, structures depicted herein are also meant to include all isomeric (e.g., enantiomeric, diastereomeric, geometric, conformational, and rotational) forms of the structure. For example, the R and S configurations for each asymmetric center, (Z) and (E) double bond isomers, and (Z) and (E) conformational isomers are included in this invention. As would be understood to one skilled in the art, a substituent can freely rotate around any rotatable bonds. For example, a substituent drawn as

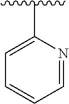 [SMILES: *c1ccccn1]

also represents

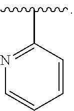 [SMILES: *c1ccccn1]

Therefore, single stereochemical isomers as well as enantiomeric, diastereomeric, geometric, conformational, and rotational mixtures of the present compounds are within the scope of the invention.

Unless otherwise indicated, all tautomeric forms of the compounds of the invention are within the scope of the invention.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C— or ¹⁴C-enriched carbon are within the scope of this invention. Such compounds are useful, for example, as analytical tools or probes in biological assays.

### Pharmaceutically Acceptable Salts

The compounds of this invention can exist in free form for treatment, or where appropriate, as a pharmaceutically acceptable salt.

A “pharmaceutically acceptable salt” means any salt or salt of an ester of a compound of this invention that, upon administration to a recipient, is capable of providing, either directly or indirectly, a compound of this invention or an inhibitorily active metabolite or residue thereof. As used herein, the term “inhibitorily active metabolite or residue thereof” means that a metabolite or residue thereof is also an inhibitor of a choline kinase.

In some embodiments, said salt is nontoxic.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. These salts can be prepared in situ during the final isolation and purification of the compounds. Acid addition salts can be prepared by 1) reacting the purified compound in its free-based form with a suitable organic or inorganic acid and 2) isolating the salt thus formed.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Base addition salts can be prepared by 1) reacting the purified compound in its acid form with a suitable organic or inorganic base and 2) isolating the salt thus formed. Salts derived from appropriate bases include alkali metal (e.g., sodium, lithium, and potassium), alkaline earth metal (e.g., magnesium and calcium), ammonium and N⁺(C₁₋₄alkyl)₄ salts. This invention also envisions the quaternization of any basic nitrogen-containing groups of the compounds disclosed herein. Water or oil-soluble or dispersible products may be obtained by such quaternization.

Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. Other acids and bases, while not in themselves pharmaceutically acceptable, may be employed in the preparation of salts useful as intermediates in obtaining the compounds of the invention and their pharmaceutically acceptable acid or base addition salts.

### ABBREVIATIONS

The following abbreviations are used:
DMSO dimethyl sulfoxide
TCA trichloroacetic acid
ATP adenosine triphosphate
BSA bovine serum albumin
DTT dithiothreitol
MOPS 4-morpholinepropanesulfonic acid
NMR nuclear magnetic resonance
HPLC high performance liquid chromatography
LCMS liquid chromatography-mass spectrometry
TLC thin layer chromatography
Rt retention time

In one embodiment of the invention, Q¹ is a 5 or 6 membered aromatic ring having 0-2 heteroatoms selected from nitrogen or sulfur; wherein Q¹ is optionally substituted with p occurrences of J¹ and is optionally fused with Q². In another example, Q¹ is not fused to Q². In yet another embodiment, Q¹ is phenyl. In another embodiment Q¹ is thiazolyl. In some embodiments, Q¹ is pyridinyl. In another embodiment, Q¹ is selected from phenyl, thiazolyl, or pyridinyl.

In another embodiment, Q¹ is independently selected from the following:

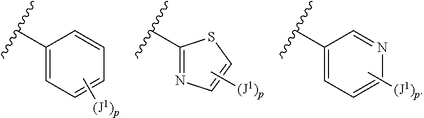

In another embodiment, Q¹ is a 5 membered aromatic ring, wherein Q¹ is optionally substituted with p occurrences of J¹. In some embodiments, Q¹ is

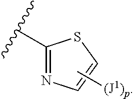

In another embodiment, Q¹ is a 6 membered aromatic ring having 0-1 heteroatoms selected from nitrogen or oxygen; wherein Q¹ is substituted with p occurrences of J¹. In other embodiments, Q¹ is independently selected from phenyl or pyridinyl. In some embodiments, Q¹ is independently selected from:

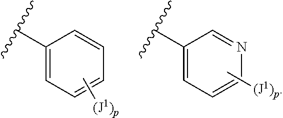

In some embodiments, J¹ is independently NR²R³, Cl, F, Br, or O(C₁₋₄aliphatic), wherein R² and R³ are C₁₋₆alkyl. In another embodiment, J¹ is ethyl or tert-butyl. In some embodiments, p is 0-2; in some embodiments, p is 0-1; in some embodiments, p is 2; in some embodiments p is 1; and in some embodiments p is 0.

In yet another embodiment, J¹ is NR²R³, wherein R² and R³ taken together with the nitrogen to which they are bound, form a five-membered heterocyclic ring. In other embodiments, J¹ is a pyrrolidinyl.

In yet another embodiment, J¹ is NR²R³, wherein R² and R³ taken together with the nitrogen to which they are bound, form a six-membered heterocyclic ring. In other embodiments, J¹ is a piperidinyl.

In some embodiments, Q² is fused to Q¹. In another embodiment, Q² is a 5 or 6 membered aromatic ring. In yet another embodiment, Q² is unsubstituted benzene.

In another embodiment, Q² is a 5 or 6 membered non-aromatic ring. In some embodiments, Q² is a 5 or 6 membered non-aromatic ring having 1-2 heteroatoms selected from nitrogen or oxygen. In some embodiments, Q² is substituted benzene. In another embodiment, Q² is dioxolyl. In yet another embodiment, Q² is pyrrolidinyl. In yet another embodiment, Q² is morpholinyl. In other embodiments, Q² is a piperazinyl. In some embodiments, Q² is independently selected from benzene, pyrrolidinyl, morpholinyl, piperazinyl, or dioxolyl. In yet another embodiment, Q² fused to Q¹ is selected from:

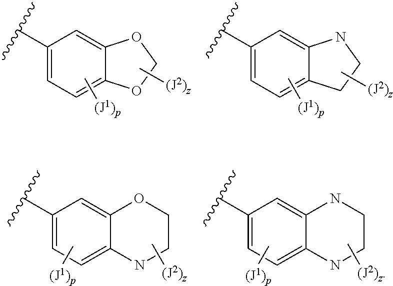

In another embodiment, Q² is independently selected from pyrrolidinyl or morpholinyl. In yet another embodiment, Q² fused to Q¹ is selected from the following:

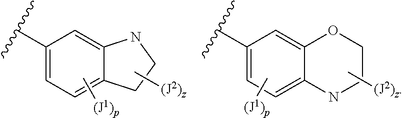

In yet another embodiment, J² is C₁₋₃ alkyl. In other embodiments, z is 0-2, in some embodiments, z is 0-1; in some embodiments z is 1; and in some embodiments, z is 0.

In some embodiments, n is 0-3; in some embodiments, n is 0-2, in some embodiments, n is 0-1, and in other embodiments, n is 0.

In some embodiments, the variables are as depicted in the compounds of Table 1.

In some embodiments, the compounds of this invention are represented in Table 1.

TABLE 1

| | |
|---|---|
| 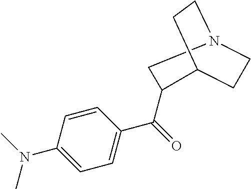 [SMILES: CN(C)c1ccc(C(=O)C2CN3CCC2CC3)cc1] | I-1 |
| 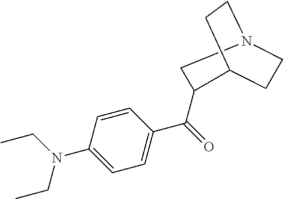 [SMILES: CCN(CC)c1ccc(C(=O)C2CN3CCC2CC3)cc1] | I-2 |
| 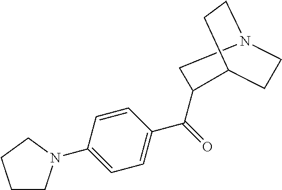 [SMILES: O=C(c1ccc(N2CCCC2)cc1)C1CN2CCC1CC2] | I-3 |
| 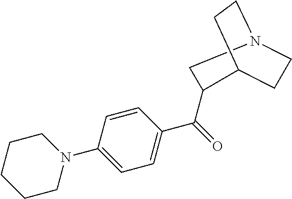 [SMILES: O=C(c1ccc(N2CCCCC2)cc1)C1CN2CCC1CC2] | I-4 |
| 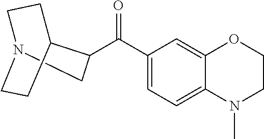 [SMILES: CN1CCOc2cc(C(=O)C3CN4CCC3CC4)ccc21] | I-5 |
| 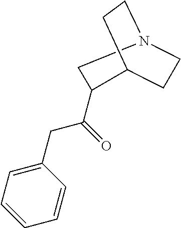 [SMILES: O=C(Cc1ccccc1)C1CN2CCC1CC2] | I-6 |
| 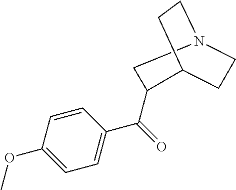 [SMILES: COc1ccc(C(=O)C2CN3CCC2CC3)cc1] | I-7 |
| 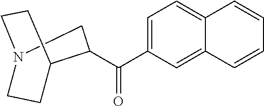 [SMILES: O=C(c1ccc2ccccc2c1)C1CN2CCC1CC2] | I-8 |
| 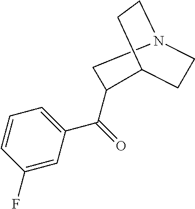 [SMILES: O=C(c1cccc(F)c1)C1CN2CCC1CC2] | I-9 |
| 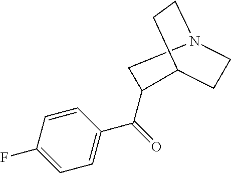 [SMILES: O=C(c1ccc(F)cc1)C1CN2CCC1CC2] | I-10 |
| 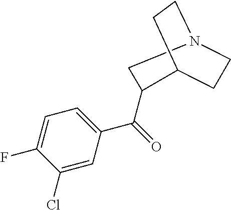 [SMILES: O=C(c1ccc(F)c(Cl)c1)C1CN2CCC1CC2] | I-11 |
| 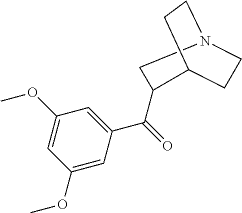 [SMILES: COc1cc(OC)cc(C(=O)C2CN3CCC2CC3)c1] | I-12 |
| 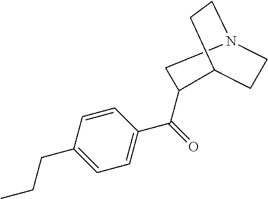 [SMILES: CCCc1ccc(C(=O)C2CN3CCC2CC3)cc1] | I-13 |
| 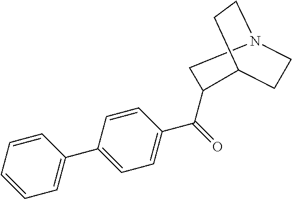 [SMILES: O=C(c1ccc(-c2ccccc2)cc1)C1CN2CCC1CC2] | I-14 |
| 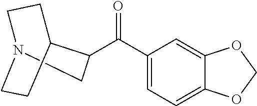 [SMILES: O=C(c1ccc2c(c1)OCO2)C1CN2CCC1CC2] | I-15 |
| 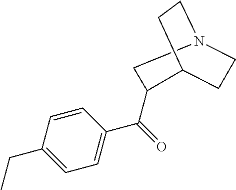 [SMILES: CCc1ccc(C(=O)C2CN3CCC2CC3)cc1] | I-16 |
| 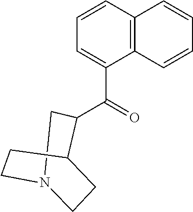 [SMILES: O=C(c1cccc2ccccc12)C1CN2CCC1CC2] | I-17 |
| 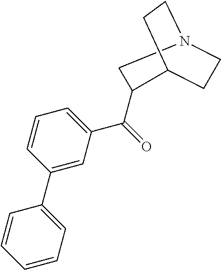 [SMILES: O=C(c1cccc(-c2ccccc2)c1)C1CN2CCC1CC2] | I-18 |
| 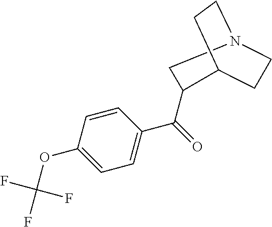 [SMILES: O=C(c1ccc(OC(F)(F)F)cc1)C1CN2CCC1CC2] | I-19 |
| 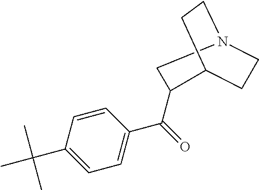 [SMILES: CC(C)(C)c1ccc(C(=O)C2CN3CCC2CC3)cc1] | I-20 |
| 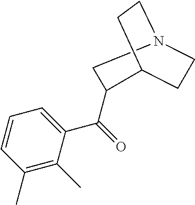 [SMILES: Cc1cccc(C(=O)C2CN3CCC2CC3)c1C] | I-21 |
| 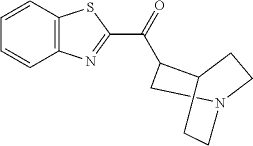 [SMILES: O=C(c1nc2ccccc2s1)C1CN2CCC1CC2] | I-22 |
| 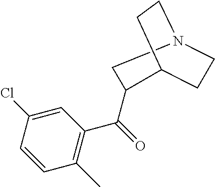 [SMILES: Cc1ccc(Cl)cc1C(=O)C1CN2CCC1CC2] | I-23 |
| 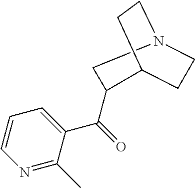 [SMILES: Cc1ncccc1C(=O)C1CN2CCC1CC2] | I-24 |
| 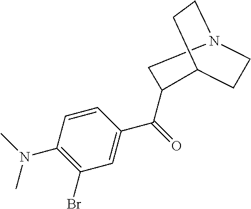 [SMILES: CN(C)c1ccc(C(=O)C2CN3CCC2CC3)cc1Br] | I-25 |
| 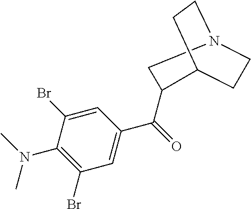 [SMILES: CN(C)c1c(Br)cc(C(=O)C2CN3CCC2CC3)cc1Br] | I-26 |
| 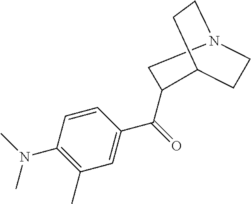 [SMILES: Cc1cc(C(=O)C2CN3CCC2CC3)ccc1N(C)C] | I-27 |
| 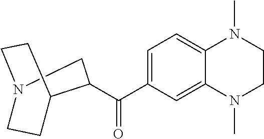 [SMILES: CN1CCN(C)c2cc(C(=O)C3CN4CCC3CC4)ccc21] | I-28 |
| 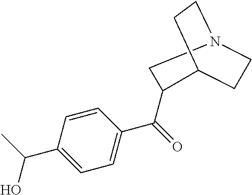 [SMILES: CC(O)c1ccc(C(=O)C2CN3CCC2CC3)cc1] | I-29 |
| 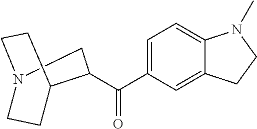 [SMILES: CN1CCc2cc(C(=O)C3CN4CCC3CC4)ccc21] | I-30 |
| 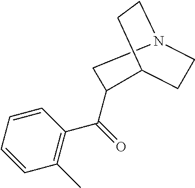 [SMILES: Cc1ccccc1C(=O)C1CN2CCC1CC2] | I-31 |
| 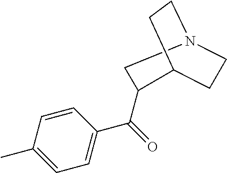 [SMILES: Cc1ccc(C(=O)C2CN3CCC2CC3)cc1] | I-32 |
| 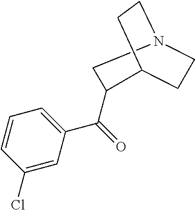 [SMILES: O=C(c1cccc(Cl)c1)C1CN2CCC1CC2] | I-33 |
| 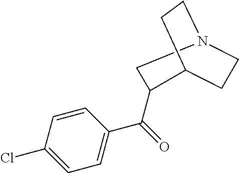 [SMILES: O=C(c1ccc(Cl)cc1)C1CN2CCC1CC2] | I-34 |
| 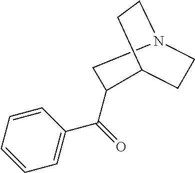 [SMILES: O=C(c1ccccc1)C1CN2CCC1CC2] | I-35 |
| 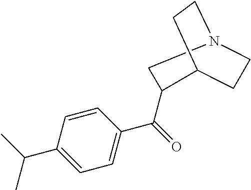 [SMILES: CC(C)c1ccc(C(=O)C2CN3CCC2CC3)cc1] | I-36 |

### General Synthetic Methodology

The compounds of this invention may be prepared in light of the specification using steps generally known to those of ordinary skill in the art. Those compounds may be analyzed by known methods, including but not limited to LCMS (liquid chromatography mass spectrometry) and NMR (nuclear magnetic resonance). It should be understood that the specific conditions shown below are only examples, and are not meant to limit the scope of the conditions that can be used for making compounds of this invention. Instead, this invention also includes conditions that would be apparent to those skilled in that art in light of this specification for making the compounds of this invention. Unless otherwise indicated, all variables in the following schemes are as defined herein

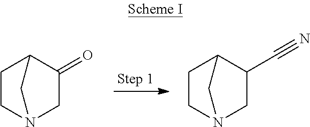 [SMILES: O=C1CN2CCC1C2 ; N#CC1CN2CCC1C2]

Scheme I above illustrates a synthetic route for preparing a quinuclidine-3-carbonitrile, which may be used as a starting material for the synthesis of the compounds of Formula I. In Step 1, a mixture of quinuclidin-3-one hydrochloride (12.58 g, 77.85 mmol), TosMic (19.75 g, 101.2 mmol), anhydrous EtOH (7.8 mL) and anhydrous 1,2-dimethoxyethane (600 mL) was cooled in an ice bath. Solid KO^{t}Bu (32.33 g, 288.1 mmol) was added portion wise over 20 minutes maintaining the temperature between 5-10° C. After complete addition, the ice bath was removed and the mixture was heated to 45.6° C. (internal) for 18 hours. After this time, the reaction mixture was allowed to cool to ambient and the solids were removed by filtration and washed with DME (300 mL). The filtrate was concentrated in vacuo and the residue was re-dissolved in a minimum amount of 2% MeOH/EtOAc and filtered through a pad of neutral alumina, eluting with more 2% MeOH/EtOAc. The filtrate was concentrated in vacuo. The residue was re-purified by column chromatography (alumina column, eluting with 0 to 2% MeOH/EtOAc) to give the title product as a light brown semi-solid (7.82 g, 74% Yield). ¹H NMR (400 MHz, CDCl₃) δ 1.42-1.71 (m, 3H); 1.90-2.03 (m, 1H); 2.08-2.13 (m, 1H); 2.64-2.70 (m, 1H); 2.73-2.94 (m, 4H); 2.96-3.06 (m, 1H); 3.20-3.28 (m, 1H).

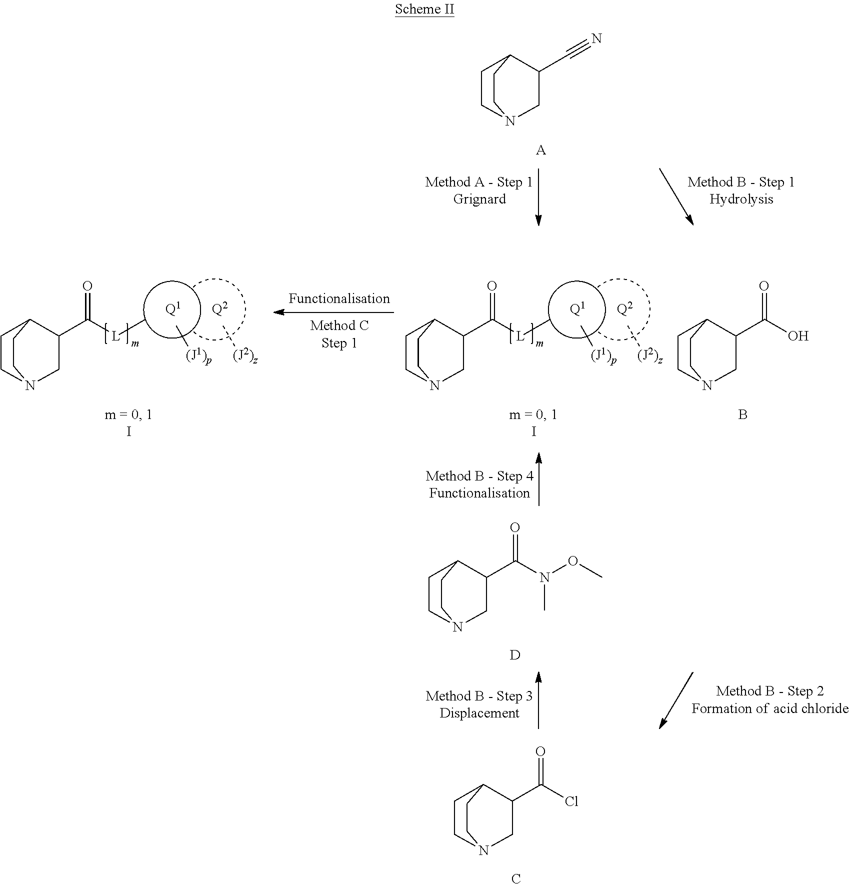

Scheme II above illustrates general methodologies for preparing the compounds of Formula I by utilizing the quinuclidine-3-carbonitrile prepared in Scheme I. It is appreciated that the synthetic routes (i.e., Methods A-C) described in Scheme II above are known to those skilled in the art.

In Method A, the quinuclidine-3-carbonitrile is reacted with an organomagnesium halide having the formula Ar—Mg—X, wherein Ar is a substituted or unsubstituted aromatic compound and X is a halide, to form Compound I. Examples of this synthetic route are provided in Examples 1-3 below.

In Method B, the quinuclidine-3-carbonitrile is hydrolyzed to form compound B, which includes a carboxylic acid substitutent at the 3 position of the quinuclidine ring. Compound B is then treated with a chlorinating agent, e.g., SOCl₂ or (COCl)₂ (see Example 4 below), to synthesize Compound C, which includes a acyl chloride at the 3 position of the quinuclidine ring. Compound C is treated with N,O-dimethylhydroxylamine, which displaces the chloride from the carbonyl carbon to form Compound D. An additional displacement reaction is performed on Compound D to synthesize Compound I. An example of this synthetic route is provided in Example 4 below.

In Method C, Compound I may be functionalized to synthesize compounds of the present invention. An example of this synthetic route is provided in Example 5 below.

Compounds of Formula I may also be prepared using any one of the intermediates described in Scheme II or the Examples provided below. Accordingly, this invention also provides a process for preparing a compound of this invention.

One embodiment of this invention provides a process for preparing a compound of formula I:

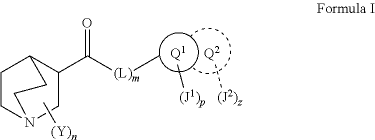

wherein L, m, Y, n, Q¹, Q², J¹, J², z and p are as defined herein, comprising reacting a compound of formula 2-a:

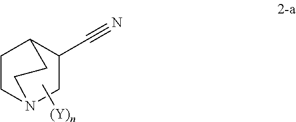

with a compound of formula i,

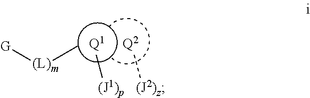

under suitable conditions to generate a nucleophilic addition reaction, wherein G is a metal or metal halide.

Organometallic compounds (e.g., organomagnesium halides and organolitium compound) are commonly associated with nucleophilic addition reactions. Suitable conditions to generate a nucleophilic addition reaction are known to those skilled in the art. For example, the process described above may be generated by combining a compound of formula 2-a with a compound of formula i in toluene, then subsequently heating the reaction mixture. Other examples of suitable nucleophilic addition conditions may be found in Solomons, T. W. Graham; Fryhle, Craig B., “Organic Chemistry”, 9^{th} edition, John Wiley & Sons, Inc. 2007.

Another embodiment of the invention provides a process for preparing a compound of Formula I:

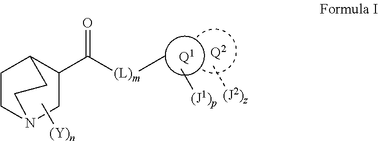

wherein L, m, Y, n, Q¹, Q², J¹, J², z and p are as defined herein, comprising:
a) reacting a compound of formula 3-a:

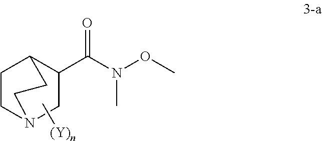

with a compound of formula iii:

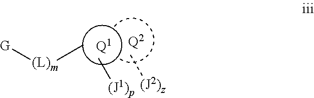

under suitable conditions to produce a displacement reaction, wherein G is lithium or a metal halide;
b) functionalizing the product of step a) to form a compound of formula I.

Suitable displacement conditions are known to those skilled in the art. For example, a compound of formula I may be produced by reacting a compound of formula 3-a with a compound of formula iii under a nitrogen atmosphere in the presence of tetrahydrofuran (THF) or dioxane. Other examples of suitable displacement conditions may be found in Solomons, T. W. Graham; Fryhle, Craig B., “Organic Chemistry”, 9^{th} edition, John Wiley & Sons, Inc. 2007.

In another example, the process further comprises reacting a compound of formula 3-b:

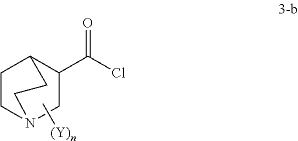

with a compound of formula iv:

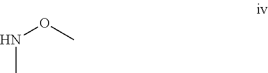

under suitable displacement conditions to form the compound of formula 3-a, described above. As stated above, suitable displacement conditions are known to those skilled in the art. For example, a compound of formula 3-a may be produced by reacting a compound of formula 3-b with a compound of formula iv in the presence of a base and an aprotic solvent. Examples of suitable bases may include, without limitation, diethyl amine, diisopropyl ethyl amine, or N-methyl morpholine. Examples of suitable aprotic solvents include, without limitation, THF, dioxane, acetonitrile, or CH₂Cl₂.

In yet another example, the process further comprises reacting a compound of formula 3-c:

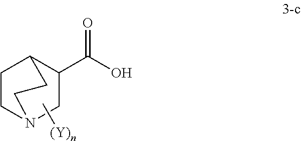

under suitable conditions to form the acid halide of formula 3-b. Suitable conditions to form the acid halide are known to those skilled in the art. For example, a compound of formula 3b may be produced by combining a compound of formula 3-c with a chlorinating agent, e.g., SOCl₂ or (COCl)₂, then heating the reaction mixture.

In yet another example, the process further comprises reacting a compound of formula 3-d:

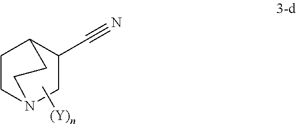

under suitable hydrolytic conditions to form the compound of formula 3-c.

Suitable hydrolytic conditions are known to those skilled in the art. For example, a compound of formula 3-c may be produced by refluxing a compound of formula 3-d with an aqueous acid, e.g., aqueous HCl, aqueous H₂SO₄. Alternatively, a formula of formula 3-c may be produced by refluxing a compound of formula 3-d with aqueous alkali, e.g., sodium hydroxide or potassium hydroxide.

### Compound Uses

One aspect of this invention provides compounds that are inhibitors of choline kinase, and thus are useful for treating or lessening the severity of a disease, condition, or disorder, wherein choline kinase is implicated in the disease, condition, or disorder.

Another aspect of this invention provides compounds that are useful for the treatment of diseases, disorders, and conditions characterized by excessive or abnormal cell proliferation. Such diseases include a proliferative or hyperproliferative disease, and a neurodegenerative disease.

Examples of proliferative and hyperproliferative diseases include, without limitation, cancer and myeloproliferative disorders.

The term “cancer” includes, but is not limited to the following cancers. Oral: buccal cavity, lip, tongue, mouth, pharynx; Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal; rectum, Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma] hairy cell; lymphoid disorders; Skin: malignant melanoma, basal cell carcinoma, squamous cell carcinoma, Karposi's sarcoma, keratoacanthoma, moles dysplastic nevi, lipoma, angioma, dermatofibroma, keloids, psoriasis, Thyroid gland: papillary thyroid carcinoma, follicular thyroid carcinoma, undifferentiated thyroid cancer, medullary thyroid carcinoma, multiple endocrine neoplasia type 2A, multiple endocrine neoplasia type 2B, familial medullary thyroid cancer, pheochromocytoma, paraganglioma; and Adrenal glands: neuroblastoma.

Thus, the term “cancerous cell” as provided herein, includes a cell afflicted by any one of the above-identified conditions. In some embodiments, the cancer is selected from colorectal, thyroid, or breast cancer.

The term “myeloproliferative disorders”, includes disorders such as polycythemia vera, thrombocythemia, myeloid metaplasia with myelofibrosis, hypereosinophilic syndrome, juvenile myelomonocytic leukemia, systemic mast cell disease, and hematopoietic disorders, in particular, acute-myelogenous leukemia (AML), chronic-myelogenous leukemia (CML), acute-promyelocytic leukemia (APL), and acute lymphocytic leukemia (ALL).

Examples of neurodegenerative diseases include, without limitation, Alzheimer's disease.

Another aspect of this invention provides compounds that are useful for the treatment of diseases and disorders, e.g., a gastroenterological disorder, a hematological disorder, an endocrinological disorder, a urological disorder, a cardiac disorder, an autoimmune disorder, a respiratory disorder, a metabolic disorder, an inflammatory disorder, an immunologically mediated disorder, a viral disease, infectious disease, or a bone disorder.

Examples of infectious disease include, without limitation, malaria.

### Pharmaceutically Acceptable Derivatives or Prodrugs

In addition to the compounds of this invention, pharmaceutically acceptable derivatives or prodrugs of the compounds of this invention may also be employed in compositions to treat or prevent the herein identified disorders.

The compounds of this invention can also exist as pharmaceutically acceptable derivatives.

A “pharmaceutically acceptable derivative” is an adduct or derivative which, upon administration to a patient in need, is capable of providing, directly or indirectly, a compound as otherwise described herein, or a metabolite or residue thereof. Examples of pharmaceutically acceptable derivatives include, but are not limited to, esters and salts of such esters.

A “pharmaceutically acceptable derivative or prodrug” means any pharmaceutically acceptable ester, salt of an ester or other derivative or salt thereof of a compound, of this invention which, upon administration to a recipient, is capable of providing, either directly or indirectly, a compound of this invention or an inhibitorily active metabolite or residue thereof. Particularly favoured derivatives or prodrugs are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

Pharmaceutically acceptable prodrugs of the compounds of this invention include, without limitation, esters, amino acid esters, phosphate esters, metal salts and sulfonate esters.

### Pharmaceutical Compositions

The present invention also provides compounds and compositions that are useful as inhibitors of choline kinase. Another aspect of the invention relates to inhibiting choline kinase activity in a biological sample or a patient, which method comprises administering to the patient a compound of Formula I or a composition comprising said compound such as a pharmaceutically acceptable carrier, adjuvant or vehicle.

The term “pharmaceutically acceptable carrier, adjuvant, or vehicle” refers to a non-toxic carrier, adjuvant, or vehicle that may be administered to a patient, together with a compound of this invention, and which does not destroy the pharmacological activity thereof.

The pharmaceutically acceptable carrier, adjuvant, or vehicle, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention.

Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### Combination Therapies

Another aspect of this invention is directed towards a method of treating cancer in a subject in need thereof, comprising the sequential or co-administration of a compound of this invention or a pharmaceutically acceptable salt thereof, and an additional therapeutic agent.

In some embodiments, said additional therapeutic agent is selected from an anti-cancer agent, an anti-proliferative agent, or a chemotherapeutic agent.

In some embodiments, said additional therapeutic agent is selected from camptothecin, the MEK inhibitor: U0126, a KSP (kinesin spindle protein) inhibitor, adriamycin, interferons, and platinum derivatives, such as Cisplatin.

In other embodiments, said additional therapeutic agent is selected from taxanes; inhibitors of bcr-abl (such as Gleevec, dasatinib, and nilotinib); inhibitors of EGFR (such as Tarceva and Iressa); DNA damaging agents (such as cisplatin, oxaliplatin, carboplatin, topoisomerase inhibitors, and anthracyclines); and antimetabolites (such as AraC and 5-FU).

In yet other embodiments, said additional therapeutic agent is selected from camptothecin, doxorubicin, idarubicin, Cisplatin, taxol, taxotere, vincristine, tarceva, the MEK inhibitor, U0126, a KSP inhibitor, vorinostat, Gleevec, dasatinib, and nilotinib.

In another embodiment, said additional therapeutic agent is selected from Her-2 inhibitors (such as Herceptin); HDAC inhibitors (such as vorinostat), VEGFR inhibitors (such as Avastin), c-KIT and FLT-3 inhibitors (such as sunitinib), BRAF inhibitors (such as Bayer's BAY 43-9006) MEK inhibitors (such as Pfizer's PD0325901); and spindle poisons (such as Epothilones and paclitaxel protein-bound particles (such as Abraxane®).

Other therapies or anticancer agents that may be used in combination with the inventive agents of the present invention include surgery, radiotherapy (in but a few examples, gamma-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), Gleevec™, adriamycin, dexamethasone, and cyclophosphamide.

A compound of the instant invention may also be useful for treating cancer in combination with any of the following therapeutic agents: abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexylen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (Masterone Injection®); Elliott's B Solution (Elliott's B Solution®); epirubicin (Ellence®); Epoetin alfa (Epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek); Rituximab (Rituxan); sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); zoledronate (Zometa®) and vorinostat (Zolinza®).

For a comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

Another aspect of this invention is directed towards a method of treating malaria in a subject in need thereof, comprising the sequential or co-administration of a compound of this invention or a pharmaceutically acceptable salt thereof, and an additional therapeutic agent.

In another aspect of the invention, said additional therapeutic agent is an anti-malarial agent.

Examples of anti-malarial agents include, without limitation, treatments for malaria, such as atovaquone-proguanil (Malarone™), artemther-lumefantrine (Coartem™), quinine sulfate, doxycycline, tetracycline, clindamycin, mefloquine (Larium™), chloroquine phosphate (Aralen™), hydroxychloroquine (Plaquenil™), primaquine phosphate, quinidine gluconate, pyrimethamide, sulfadioxine, sulfonamides, proguanil, and Halofantrine™.

Another embodiment provides a simultaneous, separate or sequential use of a combined preparation.

Compositions for Administration into a Subject

The kinase inhibitors or pharmaceutical salts thereof may be formulated into pharmaceutical compositions for administration to animals or humans. These pharmaceutical compositions, which comprise an amount of the inhibitor effective to treat or prevent a kinase-mediated condition and a pharmaceutically acceptable carrier, are another embodiment of the present invention. In some embodiments, said kinase-mediated condition is a choline kinase-mediated condition.

The term “choline kinase mediated condition”, as used herein means any disease state or other deleterious condition in which choline kinase is known to play a role. The term “choline kinase mediated condition” or “disease” also means those diseases or conditions that are alleviated by treatment with choline kinase inhibitor. Such conditions include malaria and cancer.

The exact amount of compound required for treatment will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

In some embodiments, these compositions optionally further comprise one or more additional therapeutic agents.

For example, chemotherapeutic agents or other anti-proliferative agents may be combined with the compounds of this invention to treat proliferative diseases and cancer.

Examples of known agents with which these compositions can be combined are listed above under the “Combination Therapies” section and also throughout the specification.

### Modes of Administration and Dosage Forms

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The compositions of the present invention may be administered orally, parenterally, by inhalation spray, topically, rectally, nasally, buccally, vaginally or via an implanted reservoir. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, aqueous suspensions or solutions. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include, but are not limited to, cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of kinase inhibitor that may be combined with the carrier materials to produce a single dosage form will vary depending upon the host treated, the particular mode of administration. Preferably, the compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of inhibitor will also depend upon the particular compound in the composition.

Administering with Another Agent

Depending upon the particular choline kinase-mediated conditions to be treated or prevented, additional drugs, which are normally administered to treat or prevent that condition, may be administered together with the compounds of this invention.

Those additional agents may be administered separately, as part of a multiple dosage regimen, from the kinase inhibitor-containing compound or composition. Alternatively, those agents may be part of a single dosage form, mixed together with the kinase inhibitor in a single composition.

Another aspect of this invention is directed towards a method of treating cancer in a subject in need thereof, comprising the sequential or co-administration of a compound of this invention or a pharmaceutically acceptable salt thereof, and an anti-cancer agent. In some embodiments, said anti-cancer agent is selected from camptothecin, doxorubicin, idarubicin, Cisplatin, taxol, taxotere, vincristine, tarceva, the MEK inhibitor, U0126, a KSP inhibitor, or vorinostat.

### Biological Samples

As inhibitors of kinases, the compounds and compositions of this invention are also useful in biological samples. One aspect of the invention relates to inhibiting protein kinase activity in a biological sample, which method comprises contacting said biological sample with a compound of formula I or a composition comprising said compound. The term “biological sample”, as used herein, means an in vitro or an ex vivo sample, including, without limitation, cell cultures or extracts thereof biopsied material obtained from a mammal or extracts thereof and blood, saliva, urine, feces, semen, tears, or other body fluids or extracts thereof. In some embodiments, said kinase is choline kinase. More specifically, said kinase may be choline kinase alpha (ChoKα) or choline kinase beta (ChoKβ).

Inhibition of kinase activity in a biological sample is useful for a variety of purposes that are known to one of skill in the art. Examples of such purposes include, but are not limited to, blood transfusion, organ-transplantation, and biological specimen storage.

### Study of Kinases

Another aspect of this invention relates to the study of kinases (such as choline kinase) in biological and pathological phenomena; the study of intracellular signal transduction pathways mediated by such kinases; and the comparative evaluation of new kinase inhibitors. Examples of such uses include, but are not limited to, biological assays such as enzyme assays and cell-based assays.

The activity of the compounds as kinase inhibitors may be assayed in vitro, in vivo or in a cell line. In vitro assays include assays that determine inhibition of either the kinase activity or ATPase activity of the activated kinase. Alternate in vitro assays quantitate the ability of the inhibitor to bind to the kinase and may be measured either by radiolabelling the inhibitor prior to binding, isolating the inhibitor/kinase complex and determining the amount of radiolabel bound, or by running a competition experiment where new inhibitors are incubated with the kinase bound to known radioligands. Detailed conditions for assaying a compound utilized in this invention as an inhibitor of choline kinase is set forth in the Examples below.

Another aspect of the invention provides a method for modulating enzyme activity by contacting a compound of formula I with a choline kinase.

### Methods of Treatment

In one aspect, the present invention provides a method for treating or lessening the severity of a disease, condition, or disorder where a kinase is implicated in the disease state. In another aspect, the present invention provides a method for treating or lessening the severity of a kinase disease, condition, or disorder where inhibition of enzymatic activity is implicated in the treatment of the disease. In another aspect, this invention provides a method for treating or lessening the severity of a disease, condition, or disorder with compounds that inhibit enzymatic activity by binding to the kinase. Another aspect provides a method for treating or lessening the severity of a kinase disease, condition, or disorder by inhibiting enzymatic activity of the kinase with a kinase inhibitor.

In some embodiments, said kinase inhibitor is a choline kinase inhibitor. More specifically, said kinase inhibitor is a ChoKα inhibitor.

One aspect of the invention relates to a method of inhibiting kinase activity in a patient, which method comprises administering to the patient a compound of formula I, or a composition comprising said compound.

In some embodiments, said method is used to treat or prevent a condition selected from cancer, a proliferative disorder, a gastroenterological disorder, a hematological disorder, an endocrinological disorder, a urological disorder, a cardiac disorder, a neurodegenerative disorder, an autoimmune disorder, a respiratory disorder, a metabolic disorder, an inflammatory disorder, an immunologically mediated disorder, a viral disease, infectious disease, or a bone disorder. In other embodiments, said condition is selected from cancer. In other embodiments, said condition is selected from malaria.

Another aspect of this invention provides a method for the treatment or lessening the severity of a disease selected from cancer, a proliferative disorder, a gastroenterological disorder, a hematological disorder, an endocrinological disorder, a urological disorder, a cardiac disorder, a neurodegenerative disorder, an autoimmune disorder, a respiratory disorder, a metabolic disorder, an inflammatory disorder, an immunologically mediated disorder, a viral disease, infectious disease, or a bone disorder. comprising administering an effective amount of a compound, or a pharmaceutically acceptable composition comprising a compound, to a subject in need thereof.

In certain embodiments, an “effective amount” of the compound or pharmaceutically acceptable composition is that amount effective in order to treat said disease. The compounds and compositions, according to the method of the present invention, may be administered using any amount and any route of administration effective for treating or lessening the severity of said disease.

According to another embodiment, the invention provides methods for treating or preventing cancer, a proliferative disorder, a gastroenterological disorder, a hematological disorder, an endocrinological disorder, a urological disorder, a cardiac disorder, a neurodegenerative disorder, an autoimmune disorder, a respiratory disorder, a metabolic disorder, an inflammatory disorder, an immunologically mediated disorder, a viral disease, infectious disease, or a bone disorder comprising the step of administering to a patient one of the herein-described pharmaceutical compositions. The term “patient”, as used herein, means an animal, preferably a human.

In some embodiments, said method is used to treat or prevent a condition selected from a proliferative disorder, such as cancer, a neurodegenerative disorder, an autoimmune disorder, an inflammatory disorder, and an immunologically-mediated disorder. In some embodiments, said method is used to treat or prevent a condition selected from cancers such as cancers of the breast, colon, prostate, skin, pancreas, brain, genitourinary tract, lymphatic system, stomach, larynx and lung, including lung adenocarcinoma and small cell lung cancer; stroke, diabetes, myeloma, hepatomegaly, cardiomegaly, Alzheimer's disease, cystic fibrosis, and viral disease, or any specific disease described herein.

### EXAMPLES

The compounds of this invention may be prepared in light of the specification using steps generally known to those of ordinary skill in the art. Those compounds may be analyzed by known methods, including but not limited to LCMS (liquid chromatography mass spectrometry) and NMR (nuclear magnetic resonance). Compounds of this invention may be also tested according to these examples. It should be understood that the specific conditions shown below are only examples, and are not meant to limit the scope of the conditions that can be used for making, analyzing, or testing the compounds of this invention. Instead, this invention also includes conditions known to those skilled in that art for making, analyzing, and testing the compounds of this invention.

### HPLC Methods

As used herein, the term “Rt(min)” refers to the HPLC retention time, in minutes, associated with the compound. Unless otherwise indicated, the HPLC method utilized to obtain the reported retention time is as follows:

Column: ACE C8 column, 4.6×150 mm

Gradient: 0-100% acetonitrile+methanol 60:40 (20 mM Tris phosphate)

Flow rate: 1.5 mL/minute

Detection: 225 nm.

### HNMR Methods

¹H-NMR spectra were recorded at 400 MHz using a Bruker DPX 400 instrument.

### Mass Spectrometry Methods

### Method D

Mass spec. samples were analyzed on a MicroMass Quattro Micro mass spectrometer operated in single MS mode with electrospray ionization. Samples were introduced into the mass spectrometer using chromatography. Mobile phase for all mass spec. analyses consisted of 10 mM pH 7 ammonium acetate and a 1:1 acetonitrile-methanol mixture, column gradient conditions are 5%-100% acetonitrile-methanol over 3.5 mins gradient time and 5 mins run time on an ACE C8 3.0×75 mm column. Flow rate is 1.2 ml/min.

The compounds of formula I were prepared and analyzed as follows.

### Example 1

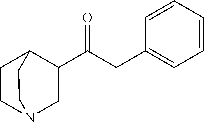 [SMILES: O=C(Cc1ccccc1)C1CN2CCC1CC2]

### 2-phenyl-1-(quinuclidin-3-yl)ethanone (Compound I-6)

### Method A

Quinuclidine-3-carbonitrile (1.11 g, 8.15 mmol) was dissolved in toluene (25 mL) under a nitrogen atmosphere. Benzylmagnesium chloride (16.81 g, 16.30 mL of 1.0 M solution in ether, 16.30 mmol) was added at ambient temperature. After 30 minutes the reaction mixture was warmed to 50° C. for an hour. After this time, water was added to the reaction mixture and stirred for an hour and then the mixture was allowed to cool to ambient temperature. The aqueous was extracted with EtOAc, and then the pH was adjusted to pH 11 using 2M NaOH. EtOAc was added, and the gelatinous mixture was filtered through celite. The layers were separated and the aqueous was extracted with EtOAc (×2) and the combined organic extracts washed with brine (×2), dried (MgSO₄) and concentrated in vacuo. The residue was purified by column chromatography (ISCO Companion™, 80 g basic alumina column, eluting with 0 to 100% EtOAc/Petroleum Ether) to give the title product as a light brown oil solid (1.21 g, 65% Yield). Some material was further purified by reverse phase preparative HPLC [Waters Sunfire C18, 10 μM, 100 Å column, gradient 10%-95% B (solvent A: 0.05% TFA in water; solvent B: CH₃CN) over 16 minutes at 25 mL/min]. The fractions were collected and passed through a bicarbonate SPE cartridge and freeze-dried to give the title compound as a yellow solid (5.73 mg). ¹H NMR (400 MHz, CDCl₃) δ 1.38 (1H, brs), 1.51 (1H, brs), 1.66 (2H, brs), 2.20 (1H, s), 2.42 (1H, vbrs), 2.80-2.90 (5H, m), 3.36 (1H, brd), 3.75 (2H, m), 7.21 (2H, m), 2.28-7.35 (3H, m); MS (ES⁺) 230.0.

The following compounds were also prepared using a sequence similar to that outlined in Example 1:

### Compound I-31: o-tolyl(quinuclidin-3-yl)methanone

¹H NMR (400 MHz, CDCl₃) δ 1.75-1.81 (m, 2H), 2.01 (t, J=2.9 Hz, 1H), 2.13 (t, J=2.8 Hz, 1H), 3.35-3.48 (m, 6H), 3.86-3.89 (m, 1H), 3.97-4.02 (m, 1H), 7.28-7.35 (m, 2H), 7.45-7.48 (m, 1H), 7.61 (d, J=7.6 Hz, 1H) and 13.02 (s, 1H) ppm; MS (ES⁺) 230.9;

### Compound I-7: (4-methoxyphenyl)-quinuclidin-3-yl-methanone

¹H NMR (400 MHz, DMSO) δ 1.23 (1H, brt), 1.36-1.40 (1H, m), 1.52-1.55 (1H, m), 1.77-1.79 (1H, m), 1.97 (1H, dd), 2.60-2.78 (4H, m), 2.87 (1H, dd), 3.16 (1H, dd), 3.59 (1H, brt), 3.85 (3H, s), 7.04 (2H, d), 7.94 (2H, d); MS (ES⁺) 246.0;

### Compound I-8: 2-naphthyl(quinuclidin-3-yl)methanone

¹H NMR (400 MHz, DMSO) δ 1.23-1.30 (2H, m), 1.43-1.49 (1H, m), 1.56-1.61 (1H, m), 1.83-1.89 (1H, m), 2.08 (1H, brs), 2.73-2.81 (3H, m), 2.99 (1H, dd), 3.20 (1H, dd), 3.82 (1H, dd), 7.61-7.69 (2H, m), 7.98-8.04 (3H, m), 8.15 (1H, d), 8.64 (1H, s); MS (ES⁺) 266.0;

### Compound I-9: (3-fluorophenyl)-quinuclidin-3-yl-methanone

¹H NMR (400 MHz, DMSO) δ 1.20-1.30 (1H, m), 1.37-1.42 (1H, m), 1.52-1.58 (1H, m), 1.99-2.02 (1H, m), 2.60-2.79 (4H, m), 2.92 (1H, dd), 3.15 (1H, dd), 3.65 (1H, brdd), 7.49 (1H, dt), 67.60 (1H, dd), 7.71 (1H, d), 7.82 (1H, d); MS (ES⁺) 234.0;

### Compound I-10: (4-fluorophenyl)-quinuclidin-3-yl-methanone

MS (ES⁺) 234.0;

### Compound I-32: p-tolyl(quinuclidin-3-yl)methanone

MS (ES⁺) 230.0;

### Compound I-33: (3-chlorophenyl)-quinuclidin-3-yl-methanone

MS (ES⁺) 250.0;

### Compound I-11: (3-chloro-4-fluoro-phenyl)-quinuclidin-3-yl-methanone

¹H NMR (400 MHz, DMSO) δ 1.19-1.23 (1H, m), 1.25-1.36 (1H, m), 1.47-1.52 (1H, m), 1.72-1.75 (1H, m), 2.59-2.75 (4H, m), 2.88 (1H, t), 3.08 (1H, dd), 3.62 (1H, brs), 7.49 (1H, dt), 7.89-7.93 (1H, m), 8.05 (1H, d); MS (ES⁺) 268.0;

### Compound I-12: (3,5-dimethoxyphenyl)-quinuclidin-3-yl-methanone

MS (ES⁺) 276.0;

### Compound I-13: (4-propylphenyl)-quinuclidin-3-yl-methanone

MS (ES⁺) 258.0;

### Compound I-14: (4-phenylphenyl)-quinuclidin-3-yl-methanone

¹H NMR (400 MHz, DMSO) δ 1.20-1.30 (1H, m), 1.39-1.46 (1H, m), 1.53-1.60 (1H, m), 1.78-1.85 (1H, m), 2.02-2.08 (1H, m), 2.60-2.68 (1H, m), 2.69-2.80 (3H, m), 2.89-2.96 (1H, m), 3.20 (1H, dd), 3.68 (1H, brt), 7.44 (1H, t), 7.52 (2H, t), 7.75 (2H, d), 7.82 (2H, d), 8.04 (2H, d); MS (ES⁺) 292.0;

### Compound I-15: 1,3-benzodioxol-5-yl(quinuclidin-3-yl)methanone

MS (ES⁺) 260.0;

### Compound I-34: (4-chlorophenyl)-quinuclidin-3-yl-methanone

¹H NMR (400 MHz, DMSO) δ 1.20-1.30 (1H, m), 1.36-1.43 (1H, m), 1.52-1.60 (1H, m), 1.77-1.82 (1H, m), 2.00-2.03 (1H, m), 2.65-2.81 (4H, m), 2.93 (1H, t), 3.17 (1H, dd), 3.65 (1H, brt), 7.60 (2H, d), 7.96 (2H, d); MS (ES⁺) 250.0;

### Compound I-16: (4-ethylphenyl)-quinuclidin-3-yl-methanone

MS (ES⁺) 244.0;

### Compound I-17: 1-naphthyl(quinuclidin-3-yl)methanone

MS (ES⁺) 266.0;

### Compound I-1: (4-dimethylaminophenyl)-quinuclidin-3-yl-methanone

(¹H NMR (400 MHz, DMSO) δ 1.22 (1H, brt), 1.42-1.46 (1H, m), 1.49-1.56 (1H, m), 1.72-1.80 (1H, m), 1.96 (1H, s), 2.60-2.64 (1H, m), 2.70-2.86 (3H, m), 3.01 (6H, s), 3.16 (1H, dd), 3.49 (1H, brt), 6.72 (1H, d), 7.79 (1H, d); MS (ES⁺) 259.0.

### Compound I-18: (3-phenylphenyl)-quinuclidin-3-yl-methanone

MS (ES⁺) 292.0;

### Compound I-19: quinuclidin-3-yl-[4-(trifluoromethoxy)phenyl]methanone

MS (ES⁺) 300.0;

### Compound I-35: phenyl(quinuclidin-3-yl)methanone

¹H NMR (400 MHz, CDCl₃) δ 1.70-1.83 (m, 2H), 2.03-2.11 (m, 1H), 2.20-2.28 (m, 1H), 2.52 (qn, J=2.9 Hz, 1H), 3.24-3.30 (m, 1H), 3.34-3.42 (m, 4H), 4.00-4.05 (m, 2H), 7.53-7.57 (m, 2H), 7.68 (s, 1H), 7.67 (t, J=7.5 Hz, 1H), 7.96-7.98 (m, 2H) and 12.75 (s, H) ppm; MS (ES⁺) 215.8;

### Compound I-20: (4-tert-butylphenyl)-quinuclidin-3-yl-methanone

¹H NMR (400 MHz, CDCl₃) δ 1.36 (s, 9H), 1.59-1.79 (m, 3H), 1.91 (s, 1H), 2.80 (s, 1H), 2.98 (m, 4H), 3.53 (s, 2H), 7.28 (s, H), 7.50 (d, J=8.5 Hz, 2H) and 7.90 (d, J=8.5 Hz, 2H) ppm; MS (ES⁺) 273.2;

### Compound I-21: (2,3-dimethylphenyl)-quinuclidin-3-yl-methanone

¹H NMR (400 MHz, CDCl₃) δ 1.78-1.87 (m, 2H), 2.00-2.10 (m, 2H), 2.30 (s, 3H), 2.34 (s, 3H), 2.41 (q, J=3.0 Hz, 1H), 3.31-3.53 (m, 5H), 3.77-3.80 (m, 1H), 4.01 (dd, J=5.7, 13.2 Hz, 1H), 6.92 (s, 1H), 7.22 (t, J=7.6 Hz, 1H), 7.34 (t, J=8.1 Hz, 2H) and 11.85 (s, 1H) ppm; MS (ES⁺) 245.2.

### Example 2

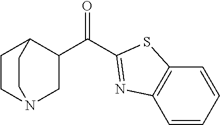 [SMILES: O=C(c1nc2ccccc2s1)C1CN2CCC1CC2]

### benzo[d]thiazol-2-yl(quinuclidin-3-yl)methanone (Compound I-22)

### Method A2

1,3-benzothiazole (106.9 mg, 86.91 μL, 0.79 mmol) was added dropwise to a solution of Chloro(ethyl)magnesium (474.4 μL of 2 M in THF, 0.9489 mmol) cooled at 5° C. The mixture was stirred for 10 minutes and then transferred to a microwave tube containing quinuclidine-3-carbonitrile (140 mg, 1.03 mmol). The mixture was heated to 120° C. for 10 minutes under microwave conditions before a 2M HCl aqueous solution was added and the mixture was further heated at 100° C. for 10 minutes under microwave conditions. After this time, the reaction mixture was basified with 6N NaOH aqueous solution, and extracted into DCM. The material was purified by reverse phase preparative HPLC [Waters Sunfire C18, 10 μM, 100 Å column, gradient 10%-95% B (solvent A: 0.05% TFA in water; solvent B: CH₃CN) over 16 minutes at 25 mL/min]. The fractions were collected to give material, which had a 85% purity level. This residue was re-subjected to reverse phase preparative HPLC (as above) using ammonium formate as the buffer. The fractions were isolated, concentrated in vacuo, re-dissolved in DCM, neutralized and re-concentrated to give the title compound as a glassy yellow solid (2.9 mg, 1.35% Yield). ¹H NMR (400 MHz, CDCl₃) δ 0.65-0.72 (m, 1H), 1.18-1.26 (m, 2H), 1.47-1.57 (m, 1H), 1.68-1.77 (m, 1H), 2.23 (td, J=5.9, 2.9 Hz, 1H), 2.61-2.69 (m, 1H), 2.78-2.88 (m, 2H), 2.96-3.02 (m, 1H), 3.26 (dd, J=6.4, 13.5 Hz, 1H), 3.83 (t, J=8.1 Hz, 1H), 7.34-7.43 (m, 2H), 7.80-7.82 (m, 1H) and 8.00 (d, J=7.7 Hz, 1H) ppm; MS (ES⁺) 273.1.

### Example 3

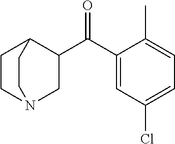 [SMILES: Cc1ccc(Cl)cc1C(=O)C1CN2CCC1CC2]

### (5-chloro-2-methylphenyl)(quinuclidin-3-yl)methanone (Compound I-23)

### Method A3

Bromo-(5-chloro-2-methyl-phenyl)magnesium (6.87 mL of 0.25 M, 1.72 mmol) was added to a solution of quinuclidine-3-carbonitrile (156 mg, 1.145 mmol) in THF (7.8 mL). The mixture was heated at reflux for 15 minutes under microwave conditions. After this time, the reaction mixture was allowed to cool to ambient and water (1 mL) was added. The mixture was heated at 80° C. for 15 minutes under microwave conditions. The aqueous was extracted with EtOAc, and then the pH was adjusted to pH 11 using 2M NaOH. EtOAc was added, and the gelatinous mixture was filtered through celite. The layers were separated and the aqueous was extracted with EtOAc (×2) and the combined organic extracts washed with brine (×2), dried (MgSO₄) and concentrated in vacuo. The residue was purified by reverse phase preparative HPLC [Waters Sunfire C18, 10 μM, 100 Å column, gradient 10%-95% B (solvent A: 0.05% TFA in water; solvent B: CH₃CN) over 16 minutes at 25 mL/min]. The fractions were collected and freeze-dried to give the TFA salt of the title compound (25.1 mg, 5.45% Yield). ¹H NMR (400 MHz, CDCl₃) 1.76-1.80 (m, 2H), 2.03-2.08 (m, 2H), 2.15 (s, 1H), 2.41 (q, J=2.8 Hz, 1H), 2.47 (d, J=4.4 Hz, 3H), 3.29 (d, J=7.0 Hz, 1H), 3.37-3.48 (m, 3H), 3.81 (s, 1H), 3.82 (dd, J=3.5, 9.7 Hz, 1H), 3.98 (dd, J=4.5, 12.9 Hz, 1H), 7.27-7.29 (m, 1H), 7.43 (dd, J=2.0, 8.2 Hz, 1H), 7.55 (d, J=2.0 Hz, 1H) and 12.92 (s, 1H) ppm; MS (ES⁺) 264.04.

The following compound was also prepared using a sequence similar to that outlined in Example 3:

### Compound I-24: (2-methyl-3-pyridyl)-quinuclidin-3-yl-methanone

¹H NMR (400 MHz, CDCl₃) δ 2.05-2.13 (m, 3H), 2.38 (q, J=3.0 Hz, 1H), 2.66 (s, 1H), 2.77 (s, 3H), 3.31 (d, J=2.3 Hz, 1H), 3.37-3.52 (m, 3H), 3.82-3.83 (m, 1H), 3.99 (s, 1H), 4.00 (dd, J=2.3, 13.4 Hz, 1H), 7.37 (dd, J=5.0, 7.9 Hz, 1H), 7.96 (dd, J=1.4, 7.9 Hz, 1H) and 8.74 (dd, J=1.6, 4.9 Hz, 1H) ppm; MS (ES⁺) 231.1.

### Example 4

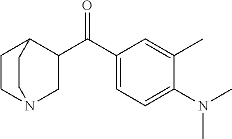 [SMILES: Cc1cc(C(=O)C2CN3CCC2CC3)ccc1N(C)C]

### (4-(dimethylamino)-3-methylphenyl)(quinuclidin-3-yl)methanone (Compound I-27)

### Method B

### Step 1

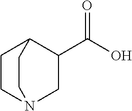 [SMILES: O=C(O)C1CN2CCC1CC2]

### (1s,4s)-quinuclidine-3-carboxylic acid hydrochloride

Quinuclidine-3-carbonitrile (120 g, 880 mmol, ca. 90% purity) was refluxed with 37% aqueous HCl (1.6 L) for 3 hours and then allowed to cool to ambient temperature for 18 hours. The reaction mixture was concentrated at 80° C. in vacuo to give a brown solid. Toluene was added and removed in vacuo. This was repeated twice (2×150 mL) to give the sub title product.

### STEP 2

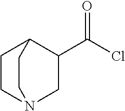 [SMILES: O=C(Cl)C1CN2CCC1CC2]

### (1s,4s)-quinuclidine-3-carbonyl chloride hydrochloride

Crude (1s,4s)-quinuclidine-3-carboxylic acid hydrochloride (max. 880 mmol) was mixed with SOCl₂ (250 mL). The mixture was heated at reflux for 1 hour. After this time, the mixture was concentration in vacuo to give an oil. Toluene was added and removed in vacuo. This was repeated twice (2×100 mL) to give the sub title product.

### STEP 3

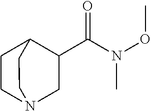

### (1s,4s)-N-methoxy-N-methylquinuclidine-3-carboxamide

Triethylamine (400 mL 2.88 moles) was added dropwise over 30 minutes to a suspension of the crude (1s,4s)-quinuclidine-3-carbonyl chloride hydrochloride (max. 880 mmol) and N,O-dimethylhydroxylamine hydrochloride (100 g, 1.03 moles) in acetonitrile (1 L) while cooling to −10° C. with an ice-acetone bath. The suspension was allowed to warm to ambient temperature over 18 hours. After this time, the suspension was filtered trough a glass filter. The salts were washed with acetonitrile (2×150 mL). The combined filtrates were concentrated in vacuo to give a dark brown oil (86 g, c.a. 89% purity). The crude was dissolved in CHCl₃ (1 L) and the resulting solution was washed with saturated aqueous K₂CO₃ (400 mL). The organic layer was separated and dried (Na₂SO₄), filtered and concentrated in vacuo to give the sub title compound (51 g, 29% Yield over 3 steps. ¹H NMR (400 MHz, CDCl₃) δ 1.29-1.39 (m, 1H); 1.57-1.65 (m, 2H); 1.77-1.86 (m, 1H); 1.94-2.04 (m, 1H); 2.70-3.02 (m, 6H); 3.16 (m, 3H); 3.20-3.29 (m, 1H); 3.69 (s, 3H); MS (ES⁺) 199.0.

### STEP 4

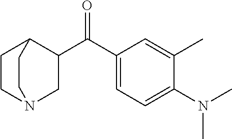 [SMILES: Cc1cc(C(=O)C2CN3CCC2CC3)ccc1N(C)C]

### (4-(dimethylamino)-3-methylphenyl)(quinuclidin-3-yl)methanone

Tert-butyllithium (707.4 mg, 1.09 mL of 1.7 M, 1.84 mmol) was added dropwise to a solution of 4-bromo-N,N,2-trimethyl-aniline (188 mg, 0.8781 mmol) in THF (10 mL) cooled at −78° C. under an nitrogen atmosphere. The reaction mixture was allowed to stir for 15 minutes. After this time, a solution of N-methoxy-N-methyl-quinuclidine-3-carboxamide (174.1 mg, 0.89 mmol) in THF (5 mL) was added dropwise over 10 minutes, and the reaction mixture was allowed to warm to ambient temperature over 18 hours. After this time, the reaction was quenched with aqueous ammonium chloride, and the mixture was concentrated in vacuo. The residue was purified by reverse phase preparative HPLC [Waters Sunfire C18, 10 μM, 100 Å column, gradient 10%-95% B (solvent A: 0.05% TFA in water; solvent B: CH₃CN) over 16 minutes at 25 mL/min]. The fractions were collected and freeze-dried to give the TFA salt of the title compound (9.4 mg, 2.74% Yield). ¹H NMR (400 MHz, MeOD) δ 1.73 (m, 2H), 2.09 (m, 2H), 2.43 (m, 1H), 2.45 (s, 6H), 2.92 (d, J=2.9 Hz, 6H), 3.28 (s, 1H), 3.32-3.44 (m, 3H), 3.79 (m, 1H), 4.00 (m, 1H), 4.19 (m, 1H), 7.21 (d, J=8.2 Hz, 1H) and 7.89 (d, J=7.9 Hz, 2H) ppm; MS (ES⁺) 273.5.

The following compounds were also prepared using a sequence similar to that outlined in Method B:

### Compound I-28: (1,4-dimethyl-2,3-dihydroquinoxalin-6-yl)-quinuclidin-3-yl-methanone

¹H NMR (400 MHz, CDCl₃) δ 1.70 (m, 1H), 1.87 (m, 1H), 2.05 (m, 1H), 2.21 (m, 1H), 2.49 (m, 1H), 2.96 (s, 3H), 3.04 (s, 3H), 3.30-3.32 (m, 3H), 3.36-3.46 (m, 4H), 3.56-3.58 (m, 2H), 3.92 (s, 1H), 4.03 (s, 1H), 4.21 (m, 2H), 6.48 (d, J=8.5 Hz, 1H), 7.25 (s, 1H) and 7.37 (dd, J=1.9, 8.5 Hz, 1H) ppm; MS (ES⁺) 300.3;

### Compound I-2: [4-(diethylamino)phenyl]-quinuclidin-3-yl-methanone

¹H NMR (400 MHz, CDCl₃) δ 1.23 (t, J=7.1 Hz, 6H), 1.71 (s, 1H), 1.87 (dd, J=2.8, 4.9 Hz, 1H), 2.05-2.09 (m, 1H), 2.22 (t, J=2.8 Hz, 1H), 2.49 (q, J=2.9 Hz, 1H), 3.28 (d, J=2.8 Hz, 1H), 3.38-3.49 (m, 4H), 3.92 (d, J=7.6 Hz, 1H), 4.06 (dd, J=5.0, 12.8 Hz, 1H), 6.70 (d, J=9.0 Hz, 2H), 7.85 (d, J=9.0 Hz, 2H) and 11.27 (s, 1H) ppm; MS (ES⁺) 287.2;

### Compound I-29: [4-(1-hydroxyethyl)phenyl]-quinuclidin-3-yl-methanone

¹H NMR (400 MHz, MeOD) δ 1.44-1.48 (m, 3H), 1.66-1.84 (m, 3H), 2.05-2.13 (m, 2H), 2.28-2.36 (m, 1H), 2.46-2.47 (m, 1H), 3.26-3.51 (m, 4H), 3.97-4.02 (m, 1H), 4.23-4.27 (m, 1H), 4.88-4.95 (m, 1H), 7.57 (d, J=8.3 Hz, 2H) and 8.05 (d, J=8.3 Hz, 2H) ppm; MS (ES⁺) 260.2;

### Compound I-3: (4-pyrrolidin-1-ylphenyl)-quinuclidin-3-yl-methanone

¹H NMR (400 MHz, CDCl₃) δ 1.22 (s, H), 1.61-1.68 (m, 1H), 1.81-1.88 (m, 1H), 1.96-2.17 (m, 6H), 2.43 (q, J=3.0 Hz, 1H), 3.15-3.23 (m, 1H), 3.28-3.42 (m, 8H), 3.82-3.86 (m, 1H), 3.92-3.97 (m, 1H), 6.56 (dd, J=2.7, 11.7 Hz, 2H), 7.84-7.87 (m, 2H) and 8.49 (s, 1H) ppm; MS (ES⁺) 285.5;

### Compound I-4: [4-(1-piperidyl)phenyl]-quinuclidin-3-yl-methanone

¹H NMR (400 MHz, CDCl₃) δ 1.73-1.77 (m, 7H), 1.81-1.88 (m, 1H), 2.03-2.11 (m, 1H), 2.19-2.26 (m, 1H), 2.49 (q, J=3.0 Hz, 1H), 3.26-3.40 (m, 1H), 3.44 (d, J=5.8 Hz, 8H), 3.91-3.94 (m, 1H), 4.06 (dd, J=5.3, 13.0 Hz, 1H), 6.97 (d, J=9.0 Hz, 2H), 7.88 (d, J=9.0 Hz, 2H) and 11.44 (s, 1H) ppm; MS (ES⁺) 300.7;

### Compound I-30: (4-methyl-2,3-dihydro-1,4-benzoxazin-7-yl)-quinuclidin-3-yl-methanone

¹H NMR (400 MHz, CDCl₃) δ 8.38 (s, 1H), 7.53 (dd, J=1.5, 8.4 Hz, 1H), 7.46 (s, 1H), 6.15 (d, J=8.4 Hz, 1H), 3.68 (dd, J=2.4, 9.7 Hz, 1H), 3.65 (s, 1H), 3.57-3.55 (m, 1H), 3.36 (t, J=8.5 Hz, 2H), 3.13-3.03 (m, 4H), 2.93-2.92 (m, 1H), 2.85 (t, J=8.4 Hz, 2H), 2.69 (s, 3H), 2.19 (q, J=3.0 Hz, 1H), 1.90 (d, J=3.1 Hz, 1H), 1.78 (s, 1H), 1.61 (t, J=2.6 Hz, 1H) and 1.41 (d, J=2.0 Hz, 1H) ppm; MS (ES⁺) 271.3;

### Compound I-5: (1-methylindolin-5-yl)-quinuclidin-3-yl-methanone

¹H NMR (400 MHz, CDCl₃) δ 11.38 (s, 1H), 7.55 (d, 1H), 7.36 (s, 1H), 7.29 (d, 1H), 4.30 (m, 2H), 4.04 (m, 1H), 3.87 (m, 1H), 3.45 (m, 6H), 3.28 (m, 1H), 3.05 (s, 3H), 2.49 (m, 1H), 2.20 (m, 1H), 2.06 (m, 1H), 1.86 (m, 1H), 1.70 (s, 1H) ppm; MS (ES⁺) 287.2.

### Example 5

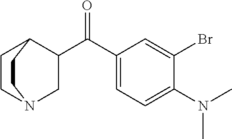

### [3-bromo-4-(dimethylamino)phenyl]-quinuclidin-3-yl-methanone (Compound I-25)

### Method C

NBS was added to a suspension of (4-dimethylaminophenyl)-quinuclidin-3-yl-methanone in PEG-400, and the reaction mixture was stirred for 20 minutes at ambient temperature. After this time, the reaction was diluted with water and extracted with EtOAc, dried (MgSO₄), filtered and concentrated in vacuo. The residue was purified by reverse phase preparative HPLC [Waters Sunfire C18, 10 μM, 100 Å column, gradient 10%-95% B (solvent A: 0.05% TFA in water; solvent B: CH₃CN) over 16 minutes at 25 mL/min]. The fractions were collected and freeze-dried to give the TFA salt of the title compound (22.6 mg, 16.2% Yield). ¹H NMR (400 MHz, CDCl₃) δ 1.77 (m, 2H), 2.11 (m, 1H), 2.27 (m, 1H), 2.49 (d, J=3.0 Hz, 1H), 2.98 (s, 6H), 3.31 (m, 1H), 3.45 (s, 4H), 3.93 (m, 1H), 4.03 (m, 1H), 6.38 (s, 2H), 7.05 (d, J=8.6 Hz, 1H), 7.84 (dd, J=2.1, 8.5 Hz, 1H), 8.14 (d, J=2.1 Hz, 1H) and 11.61 (s, 1H) ppm; MS (ES⁺) 337.0.

The following compound was also prepared using a sequence similar to that outlined in Example 5:

### Compound I-26: [3,5-dibromo-4-(dimethylamino)phenyl]-quinuclidin-3-yl-methanone

¹H NMR (400 MHz, CDCl₃) δ 1.74 (d, J=1.6 Hz, 2H), 2.08 (m, 2H), 2.48 (d, J=3.0 Hz, 1H), 2.97 (s, 6H), 3.39 (m, 5H) 3.86 (s, 1H), 3.97 (s, 1H), 8.06 (s, 2H) and 13.34 (s, 1H) ppm; MS (ES⁺) 417.0.

### Analytical Data

| | LCMS | LCMS | |
|---|---|---|---|
| Compound | (M + 1) | Rt (min) | HNMR |
| I-1 | 259 | 0.56 | (400 MHz, DMSO) 1.22 (1H, brt), 1.42-1.46 (1H, m), |
| | | | 1.49-1.56 (1H, m), 1.72-1.80 (1H, m), 1.96 (1H, s), |
| | | | 2.60-2.64 (1H, m), 2.70-2.86 (3H, m), 3.01 (6H, s), |
| | | | 3.16 (1H, dd), 3.49 (1H, brt), 6.72 (1H, d), 7.79 (1H, |
| | | | d). |
| I-2 | 287.2 | 2.37 | H NMR (400.0 MHz, CDCl3) d 1.23 (t, J = 7.1 Hz, |
| | | | 6H), 1.71 (s, 1H), 1.87 (dd, J = 2.8, 4.9 Hz, 1H), 2.05- |
| | | | 2.09 (m, 1H), 2.22 (t, J = 2.8 Hz, 1H), 2.49 (q, J = |
| | | | 2.9 Hz, 1H), 3.28 (d, J = 2.8 Hz, 1H), 3.38-3.49 (m, |
| | | | 4H), 3.92 (d, J = 7.6 Hz, 1H), 4.06 (dd, J = 5.0, 12.8 |
| | | | Hz, 1H), 6.70 (d, J = 9.0 Hz, 2H), 7.85 (d, J = 9.0 Hz, |
| | | | 2H) and 11.27 (s, 1H) ppm |
| I-3 | 285.5 | 0.66 | H NMR (400.0 MHz, CDCl3) d 1.22 (s, H), 1.61- |
| | | | 1.68 (m, 1H), 1.81-1.88 (m, 1H), 1.96-2.17 (m, |
| | | | 6H), 2.43 (q, J = 3.0 Hz, 1H), 3.15-3.23 (m, 1H), |
| | | | 3.28-3.42 (m, 8H), 3.82-3.86 (m, 1H), 3.92-3.97 |
| | | | (m, 1H), 6.56 (dd, J = 2.7, 11.7 Hz, 2H), 7.84-7.87 |
| | | | (m, 2H) and 8.49 (s, 1H) ppm |
| I-4 | 300.7 | 0.7 | H NMR (400.0 MHz, CDCl3) d 1.73-1.77 (m, 7H), |
| | | | 1.81-1.88 (m, 1H), 2.03-2.11 (m, 1H), 2.19-2.26 |
| | | | (m, 1H), 2.49 (q, J = 3.0 Hz, 1H), 3.26-3.40 (m, |
| | | | 1H), 3.44 (d, J = 5.8 Hz, 8H), 3.91-3.94 (m, 1H), |
| | | | 4.06 (dd, J = 5.3, 13.0 Hz, 1H), 6.97 (d, J = 9.0 Hz, |
| | | | 2H), 7.88 (d, J = 9.0 Hz, 2H) and 11.44 (s, 1H) ppm |
| I-5 | 287.2 | 0.54 | H NMR (400.0 MHz, CDCl3) d 11.38 (s, 1H), 7.55 |
| | | | (d, 1H), 7.36 (s, 1H), 7.29 (d, 1H), 4.30 (m, 2H), 4.04 |
| | | | (m, 1H), 3.87 (m, 1H), 3.45 (m, 6H), 3.28 (m, 1H), |
| | | | 3.05 (s, 3H), 2.49 (m, 1H), 2.20 (m, 1H), 2.06 (m, |
| | | | 1H), 1.86 (m, 1H), 1.70 (s, 1H) ppm |
| I-6 | 230 | 0.51 | (400 MHz, CDCl3) 1.38 (1H, brs), 1.51 (1H, brs), |
| | | | 1.66 (2H, brs), 2.20 (1H, s), 2.42 (1H, vbrs), 2.80- |
| | | | 2.90 (5H, m), 3.36 (1H, brd), 3.75 (2H,. m), 7.21 (2H, |
| | | | m), 2.28-7.35 (3H, m). |
| I-7 | 246 | 2.04 | (400 MHz, DMSO) 1.23 (1H, brt), 1.36-1.40 (1H, m), |
| | | | 1.52-1.55 (1H, m), 1.77-1.79 (1H, m), 1.97 (1H, dd), |
| | | | 2.60-2.78 (4H, m), 2.87 (1H, dd), 3.16 (1H, dd), 3.59 |
| | | | (1H, brt), 3.85 (3H, s), 7.04 (2H, d), 7.94 (2H, d). |
| I-8 | 266 | 0.66 | (400 MHz, DMSO) 1.23-1.30 (2H, m), 1.43-1.49 (1H, |
| | | | m), 1.56-1.61 (1H, m), 1.83-1.89 (1H, m), 2.08 (1H, |
| | | | brs), 2.73-2.81 (3H, m), 2.99 (1H, dd), 3.20 (1H, dd), |
| | | | 3.82 (1H, dd), 7.61-7.69 (2H, m), 7.98-8.04 (3H, m), |
| | | | 8.15 (1H, d), 8.64 (1H, s). |
| I-9 | 234 | 0.5 | (400 MHz, DMSO) 1.20-1.30 (1H, m), 1.37-1.42 (1H, |
| | | | m), 1.52-1.58 (1H, m), 1.99-2.02 (1H, m), 2.60-2.79 |
| | | | (4H, m), 2.92 (1H, dd), 3.15 (1H, dd), 3.65 (1H, |
| | | | brdd), 7.49 (1H, dt), 67.60 (1H, dd), 7.71 (1H, d), |
| | | | 7.82 (1H, d). |
| I-10 | 234 | 0.5 | |
| I-11 | 268 | 0.62 | (400 MHz, DMSO) 1.19-1.23 (1H, m), 1.25-1.36 (1H, |
| | | | m), 1.47-1.52 (1H, m), 1.72-1.75 (1H, m), 2.59-2.75 |
| | | | (4H, m), 2.88 (1H, t), 3.08 (1H, dd), 3.62 (1H, brs), |
| | | | 7.49 (1H, dt), 7.89-7.93 (1H, m), 8.05 (1H, d), |
| I-12 | 276 | 0.57 | |
| I-13 | 258 | 0.71 | |
| I-14 | 292 | 0.72 | (400 MHz, DMSO) 1.20-1.30 (1H, m), 1.39-1.46 (1H, |
| | | | m), 1.53-1.60 (1H, m), 1.78-1.85 (1H, m), 2.02-2.08 |
| | | | (1H, m), 2.60-2.68 (1H, m), 2.69-2.80 (3H, m), 2.89- |
| | | | 2.96 (1H, m), 3.20 (1H, dd), 3.68 (1H, brt), 7.44 (1H, |
| | | | t), 7.52 (2H, t), 7.75 (2H, d), 7.82 (2H, d), 8.04 (2H, |
| | | | d). |
| I-15 | 260 | 0.5 | |
| I-16 | 244 | 0.65 | |
| I-17 | 266 | 0.64 | |
| I-18 | 292 | 0.72 | |
| I-19 | 300 | 0.69 | |
| I-20 | 273.2 | 0.76 | H NMR (400.0 MHz, CDCl3) d 1.36 (s, 9H), 1.59- |
| | | | 1.79 (m, 3H), 1.91 (s, 1H), 2.80 (s, 1H), 2.98 (m, |
| | | | 4H), 3.53 (s, 2H), 7.28 (s, H), 7.50 (d, J = 8.5 Hz, |
| | | | 2H) and 7.90 (d, J = 8.5 Hz, 2H) ppm |
| I-21 | 245.2 | 0.61 | H NMR (400.0 MHz, CDCl3) d 1.78-1.87 (m, 2H), |
| | | | 2.00-2.10 (m, 2H), 2.30 (s, 3H), 2.34 (s, 3H), 2.41 |
| | | | (q, J = 3.0 Hz, 1H), 3.31-3.53 (m, 5H), 3.77-3.80 |
| | | | (m, 1H), 4.01 (dd, J = 5.7, 13.2 Hz, 1H), 6.92 (s, |
| | | | 1H), 7.22 (t, J = 7.6 Hz, 1H), 7.34 (t, J = 8.1 Hz, 2H) |
| | | | and 11.85 (s, 1H) ppm |
| I-22 | 273.1 | 0.61 | H NMR (400.0 MHz, CDCl3) d 0.65-0.72 (m, 1H), |
| | | | 1.18-1.26 (m, 2H), 1.47-1.57 (m, 1H), 1.68-1.77 |
| | | | (m, 1H), 2.23 (td, J = 5.9, 2.9 Hz, 1H), 2.61-2.69 |
| | | | (m, 1H), 2.78-2.88 (m, 2H), 2.96-3.02 (m, 1H), |
| | | | 3.26 (dd, J = 6.4, 13.5 Hz, 1H), 3.83 (t, J = 8.1 Hz, |
| | | | 1H), 7.34-7.43 (m, 2H), 7.80-7.82 (m, 1H) and |
| | | | 8.00 (d, J = 7.7 Hz, 1H) ppm |
| I-23 | 264.04 | 2.71 | H NMR (400.0 MHz, CDCl3) d 1.76-1.80 (m, 2H), |
| | | | 2.03-2.08 (m, 2H), 2.15 (s, 1H), 2.41 (q, J = 2.8 Hz, |
| | | | 1H), 2.47 (d, J = 4.4 Hz, 3H), 3.29 (d, J = 7.0 Hz, |
| | | | 1H), 3.37-3.48 (m, 3H), 3.81 (s, 1H), 3.82 (dd, J = |
| | | | 3.5, 9.7 Hz, 1H), 3.98 (dd, J = 4.5, 12.9 Hz, 1H), |
| | | | 7.27-7.29 (m, 1H), 7.43 (dd, J = 2.0, 8.2 Hz, 1H), |
| | | | 7.55 (d, J = 2.0 Hz, 1H) and 12.92 (s, 1H) ppm |
| I-24 | 231.1 | 0.37 | H NMR (400.0 MHz, CDCl3) d 2.05-2.13 (m, 3H), |
| | | | 2.38 (q, J = 3.0 Hz, 1H), 2.66 (s, 1H), 2.77 (s, 3H), |
| | | | 3.31 (d, J = 2.3 Hz, 1H), 3.37-3.52 (m, 3H), 3.82- |
| | | | 3.83 (m, 1H), 3.99 (s, 1H), 4.00 (dd, J = 2.3, 13.4 |
| | | | Hz, 1H), 7.37 (dd, J = 5.0, 7.9 Hz, 1H), 7.96 (dd, J = |
| | | | 1.4, 7.9 Hz, 1H) and 8.74 (dd, J = 1.6, 4.9 Hz, 1H) |
| | | | ppm |
| I-25 | 337 | 0.66 | H NMR (400.0 MHz.) d 1.77 (m, 2H), 2.11 (m, 1H), |
| | | | 2.27 (m, 1H), 2.49 (d, J = 3.0 Hz, 1H), 2.98 (s, 6H), |
| | | | 3.31 (m, 1H), 3.45 (s, 4H), 3.93 (m, 1H), 4.03 (m, |
| | | | 1H), 6.38 (s, 2H), 7.05 (d, J = 8.6 Hz, 1H), 7.84 (dd, |
| | | | J = 2.1, 8.5 Hz, 1H), 8.14 (d, J = 2.1 Hz, 1H) and |
| | | | 11.61 (s, 1H) ppm |
| I-26 | 417 | 0.81 | H NMR (400.0 MHz, CDCl3) d 1.74 (d, J = 1.6 Hz, |
| | | | 2H), 2.08 (m, 2H), 2.48 (d, J = 3.0 Hz, 1H), 2.97 (s, |
| | | | 6H), 3.39 (m, 5H) 3.86 (s, 1H), 3.97 (s, 1H), 8.06 (s, |
| | | | 2H) and 13.34 (s, 1H) ppm |
| I-27 | 273.5 | 0.64 | H NMR (400.0 MHz, MeOH) d 1.73 (m, 2H), 2.09 |
| | | | (m, 2H), 2.43 (m, 1H), 2.45 (s, 6H), 2.92 (d, J = 2.9 |
| | | | Hz, 6H), 3.28 (s, 1H), 3.32-3.44 (m, 3H), 3.79 (m, |
| | | | 1H), 4.00 (m, 1H), 4.19 (m, 1H), 7.21 (d, J = 8.2 Hz, |
| | | | 1H) and 7.89 (d, J = 7.9 Hz, 2H) ppm |
| I-28 | 300.3 | 0.58 | H NMR (400.0 MHz, CDCl3) d 1.70 (m, 1H), 1.87 |
| | | | (m, 1H), 2.05 (m, 1H), 2.21 (m, 1H), 2.49 (m, 1H), |
| | | | 2.96 (s, 3H), 3.04 (s, 3H), 3.30-3.32 (m, 3H), 3.36- |
| | | | 3.46 (m, 4H), 3.56-3.58 (m, 2H), 3.92 (s, 1H), 4.03 |
| | | | (s, 1H), 4.21 (m, 2H), 6.48 (d, J = 8.5 Hz, 1H), 7.25 |
| | | | (s, 1H) and 7.37 (dd, J = 1.9, 8.5 Hz, 1H) ppm |
| I-29 | 260.2 | 0.42 | H NMR (400.0 MHz, MeOH) d 1.44-1.48 (m, 3H), |
| | | | 1.66-1.84 (m, 3H), 2.05-2.13 (m, 2H), 2.28-2.36 |
| | | | (m, 1H), 2.46-2.47 (m, 1H), 3.26-3.51 (m, 4H), |
| | | | 3.97-4.02 (m, 1H), 4.23-4.27 (m, 1H), 4.88-4.95 |
| | | | (m, 1H), 7.57 (d, J = 8.3 Hz, 2H) and 8.05 (d, J = 8.3 |
| | | | Hz, 2H) ppm |
| I-30 | 271.3 | 0.58 | H NMR (400.0 MHz, CDCl3) d 8.38 (s, 1H), 7.53 |
| | | | (dd, J = 1.5, 8.4 Hz, 1H), 7.46 (s, 1H), 6.15 (d, J = |
| | | | 8.4 Hz, 1H), 3.68 (dd, J = 2.4, 9.7 Hz, 1H), 3.65 (s, |
| | | | 1H), 3.57-3.55 (m, 1H), 3.36 (t, J = 8.5 Hz, 2H), |
| | | | 3.13-3.03 (m, 4H), 2.93-2.92 (m, 1H), 2.85 (t, J = |
| | | | 8.4 Hz, 2H), 2.69 (s, 3H), 2.19 (q, J = 3.0 Hz, 1H), |
| | | | 1.90 (d, J = 3.1 Hz, 1H), 1.78 (s, 1H), 1.61 (t, J = 2.6 |
| | | | Hz, 1H) and 1.41 (d, J = 2.0 Hz, 1H) ppm |
| I-31 | 230.9 | 0.57 | H NMR (400.0 MHz, CDCl3) d 1.75-1.81 (m, 2H), |
| | | | 2.01 (t, J = 2.9 Hz, 1H), 2.13 (t, J = 2.8 Hz, 1H), 3.35- |
| | | | 3.48 (m, 6H), 3.86-3.89 (m, 1H), 3.97-4.02 (m, |
| | | | 1H), 7.28-7.35 (m, 2H), 7.45-7.48 (m, 1H), 7.61 |
| | | | (d, J = 7.6 Hz, 1H) and 13.02 (s, 1H) ppm |
| I-32 | 230 | 0.56 | |
| I-33 | 250 | 0.58 | |
| I-34 | 250 | 0.6 | (400 MHz, DMSO) 1.20-1.30 (1H, m), 1.36-1.43 (1H, |
| | | | m), 1.52-1.60 (1H, m), 1.77-1.82 (1H, m), 2.00-2.03 |
| | | | (1H, m), 2.65-2.81 (4H, m), 2.93 (1H, t), 3.17 (1H, |
| | | | dd), 3.65 (1H, brt), 7.60 (2H, d), 7.96 (2H, d). |
| I-35 | 215.8 | 0.49 | H NMR (400.0 MHz, CDCl3) d 1.70-1.83 (m, 2H), |
| | | | 2.03-2.11 (m, 1H), 2.20-2.28 (m, 1H), 2.52 (qn, J = |
| | | | 2.9 Hz, 1H), 3.24-3.30 (m, 1H), 3.34-3.42 (m, |
| | | | 4H), 4.00-4.05 (m, 2H), 7.53-7.57 (m, 2H), 7.68 |
| | | | (s, 1H), 7.67 (t, J = 7.5 Hz, 1H), 7.96-7.98 (m, 2H) |
| | | | and 12.75 (s, H) ppm |

### Choline Kinase Alpha Assay

The compounds of the present invention are evaluated as inhibitors of Choline Kinase Alpha using the following assays.

### Choline Kinase Alpha Inhibition Assay

An assay buffer solution was prepared which consisted of 100 mM Tris-HCl (pH 7.5), 100 mM KCl, and 10 mM MgCl₂. An enzyme buffer containing reagents to final assay concentrations of 290 μM NADH, 2.4 mM phosphoenolpyruvate, 60 μg/mL pyruvate kinase, 20 μg/mL lactate dehydrogenase, 200 μM choline chloride substrate and 20 nM Choline Kinase alpha enzyme was prepared in assay buffer. To 32 μL of this enzyme buffer, in a 96 well plate, was added 2 μL of VRT stock solution in DMSO. The mixture was allowed to equilibrate for 10 mins at 25° C. The enzyme reaction was initiated by the addition of 32 μL stock ATP solution prepared in assay buffer to a final assay concentration of 400 μM. Initial rate data was determined from the rate of change of absorbance at 340 nM (corresponding to stoichiometric consumption of NADH) using a Molecular Devices Spectramax plate reader (Sunnyvale, Calif.) over 15 mins at 25° C. For each IC₅₀ determination, 12 data points covering the VRT concentration range of 0-100 μM were obtained in duplicate (DMSO stocks were prepared from an initial 10 mM VRT stock with subsequent 1:2.5 serial dilutions). IC₅₀ values were calculated from initial rate data using the Prism software package (Prism 4.0a, Graphpad Software, San Diego, Calif.).

In general, the compounds of the present invention are effective for inhibiting Choline Kinase Alpha. Preferred compounds showed IC₅₀ values below 0.1 μM (I-1, I-3, and 1-5). Preferred compounds showed IC₅₀ values between 0.1 μM and 1 μM (I-2, I-4, I-8, I-13, I-16, I-20, I-25, I-27, I-28, I-30, and I-36). Other preferred compounds showed an IC50 value between 1 μM and 50 μM (I-6, I-7, I-9, I-10, I-11, I-12, I-14, I-15, I-17, I-18, I-19, I-21, I-22, I-23, I-24, I-26, I-29, I-31, I-32, I-33, I-34, and I-35).

### Choline Kinase Alpha Expression and Purification

hChoKα1(M1-V457) (NP_{—}001268) was codon optimized for E. coli and cloned into a modified pGEX-2T vector. Recombinant GST-tagged ChoKα1 protein was produced in E. coli strain BL21(DE3). After growing cell cultures at 37° C. until the OD₆₀₀=1, the cultures were induced with 1 mM IPTG for 16 h at 30° C. and the cells were harvested as a pellet (8500 rpm, 4° C., 20 min). The protein was purified using glutathione affinity purification followed by size exclusion via Superdex-200 26/60 (GE Healthcare). See Malito, Enrico et. al., “Journal of Molecular Biology”, Volume 364, Issue 2, pages 136-151 (November 2006).

While we have described a number of embodiments of this invention, it is apparent that our basic examples may be altered to provide other embodiments that utilize the compounds, methods, and processes of this invention. Therefore, it will be appreciated that the scope of this invention is to be defined by the appended claims rather than by the specific embodiments that have been represented by way of example herein.

We claim:

1. A compound of formula: 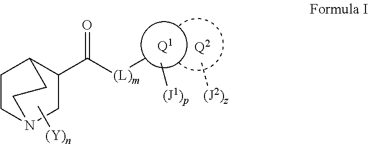
or a pharmaceutically acceptable salt thereof; wherein
Y is bonded to any carbon atom of the quinuclidine ring and is independently C₁₋₃ aliphatic, —CF₃, —CN, halo, ═O, —OH, —O(C₁₋₃aliphatic), NH₂, or NH(C₁₋₃ aliphatic);
n is 0-4;
L is a C₁₋₂ alkyl;
m is 0 or 1;
Q¹ is a 5 or 6 membered aromatic or non-aromatic ring having 0-2 heteroatoms independently selected from nitrogen, oxygen, or sulfur; wherein Q¹ is optionally substituted with p occurrences of J¹ and is optionally fused with Q²;
Q² is a 5 or 6 membered aromatic or non-aromatic ring having 0-2 heteroatoms independently selected from nitrogen, oxygen, or sulfur, wherein Q² is optionally substituted with z occurrences of J²;
J¹ is —Cl, —F, —Br, —NR²R³, —OCF₃, —O(C₁₋₄ aliphatic), -methyl, -ethyl, -tert-butyl, -propyl, —CF₃, —CN, or phenyl, wherein said J¹ is independently and optionally substituted with 1-3 occurrences of halo, —O(C₁₋₄aliphatic), —CN, or —OH;
R² is H or C₁₋₆ alkyl;
R³ is H or C₁₋₆ alkyl;
or R² and R³, taken together with the atom to which they are bound, form a 4-8 membered heterocyclic ring having 1-2 heteroatoms selected from oxygen, nitrogen, or sulfur;
p is 0, 1, 2, or 3, wherein p is not 0 when m is 0, and p is at least 2 when Q¹ is a phenyl, J¹ is Cl or methyl, and Q² is absent;
J² is C₁₋₃ alkyl, halo, or CF₃; and
z is 0, 1, 2, or 3.

2. The compound of claim 1, wherein n is 0.

3. The compound according to any one of claim 1 or 2, wherein Q¹ is independently selected from phenyl, thiazolyl, or pyridinyl.

4. The compound of claim 3, wherein Q¹ is selected from the following:

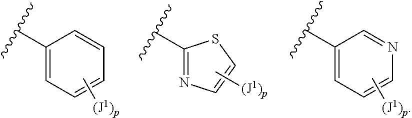

5. The compound according to any one of claims 1-3, wherein Q¹ is phenyl.

6. The compound according to any one of claims 1-5, wherein J¹ is NR²R³.

7. The compound of claim 6, wherein R² is C₁₋₆alkyl and R³ is C₁₋₆alkyl.

8. The compound according to any one of claim 6, wherein R² and R³, taken together with the nitrogen to which they are bound, form a 5 membered heterocyclic ring.

9. The compound of claim 8, wherein J¹ is pyrrolidinyl.

10. The compound according to any one of claims 1-6, wherein R² and R³, taken together with the nitrogen to which they are bound, form a 6 membered heterocyclic ring.

11. The compound of claim 10, wherein J¹ is piperidinyl.

12. The compound according to any one of claim 1 or 2, wherein J¹ is ethyl or tert-butyl.

13. The compound according to any one of claims 1-12, wherein Q² is absent.

14. The compound according to any one of claims 1-12, wherein Q¹ is fused to Q².

15. The compound of claim 14, wherein Q² is benzo.

16. The compound of claim 15, wherein Q² is fused to Q1 to form naphthalene.

17. The compound of claim 16, wherein J² is C₁₋₃ alkyl

18. The compound of claim 17, wherein J² is methyl.

19. The compound of claim 14, wherein Q² is a 5 or 6 membered non-aromatic ring having 1-2 heteroatoms selected from nitrogen or oxygen.

20. The compound of claim 19, wherein Q² is independently selected from pyrrolidinyl, morpholinyl, piperazinyl, or dioxolyl.

21. The compound of claim 20, wherein Q¹ fused to Q² forms Q¹-Q² and is selected from the following:

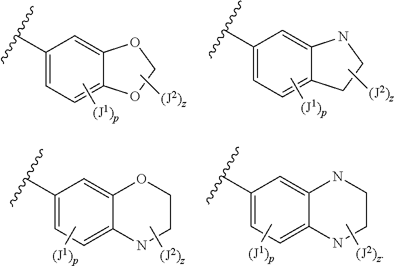

22. The compound of claim 20, wherein Q² is selected from pyrrolidinyl or morpholinyl.

23. The compound of claim 22, wherein Q² is selected from the following:

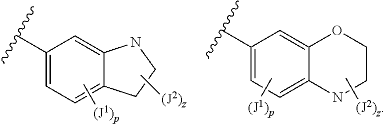

24. The compound of claim 20, wherein J² is substituted with C₁₋₃ alkyl.

25. A compound selected from the following compounds:

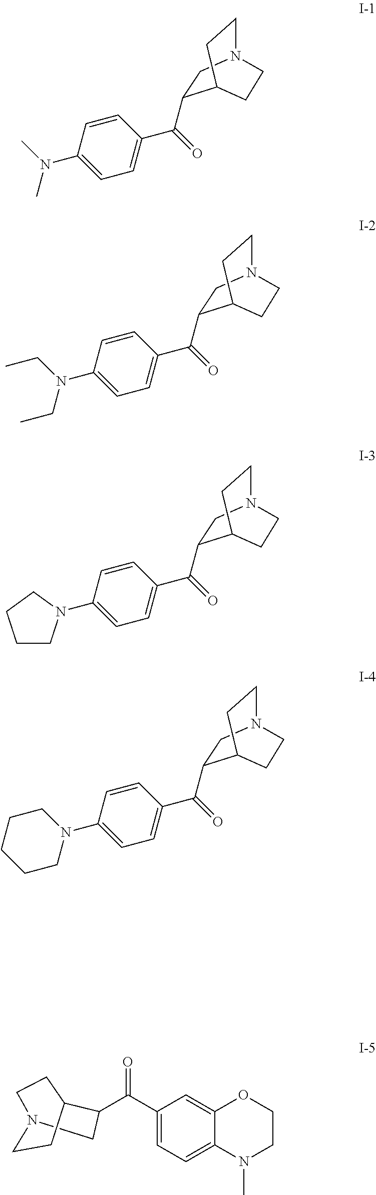 [SMILES: CN(C)c1ccc(C(=O)C2CN3CCC2CC3)cc1 ; CCN(CC)c1ccc(C(=O)C2CN3CCC2CC3)cc1 ; O=C(c1ccc(N2CCCC2)cc1)C1CN2CCC1CC2 ; O=C(c1ccc(N2CCCCC2)cc1)C1CN2CCC1CC2 ; CN1CCOc2cc(C(=O)C3CN4CCC3CC4)ccc21]

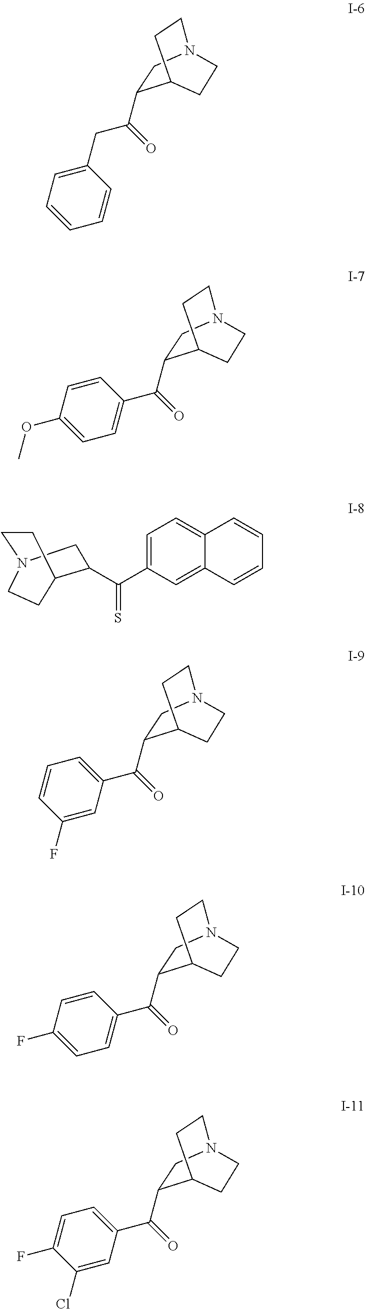 [SMILES: O=C(Cc1ccccc1)C1CN2CCC1CC2 ; COc1ccc(C(=O)C2CN3CCC2CC3)cc1 ; S=C(c1ccc2ccccc2c1)C1CN2CCC1CC2 ; O=C(c1cccc(F)c1)C1CN2CCC1CC2 ; O=C(c1ccc(F)cc1)C1CN2CCC1CC2 ; O=C(c1ccc(F)c(Cl)c1)C1CN2CCC1CC2]

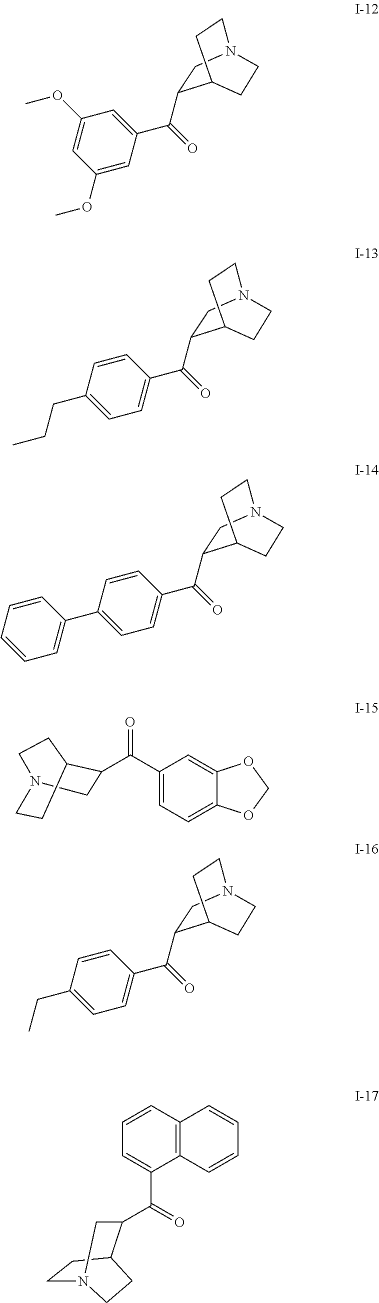 [SMILES: COc1cc(OC)cc(C(=O)C2CN3CCC2CC3)c1 ; CCCc1ccc(C(=O)C2CN3CCC2CC3)cc1 ; O=C(c1ccc(-c2ccccc2)cc1)C1CN2CCC1CC2 ; O=C(c1ccc2c(c1)OCO2)C1CN2CCC1CC2 ; CCc1ccc(C(=O)C2CN3CCC2CC3)cc1 ; O=C(c1cccc2ccccc12)C1CN2CCC1CC2]

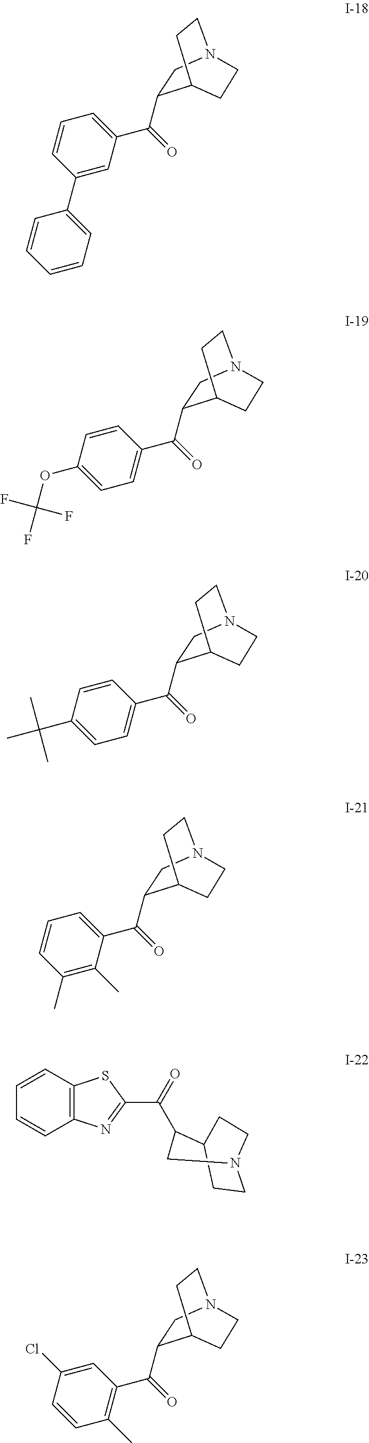 [SMILES: O=C(c1cccc(-c2ccccc2)c1)C1CN2CCC1CC2 ; O=C(c1ccc(OC(F)(F)F)cc1)C1CN2CCC1CC2 ; CC(C)(C)c1ccc(C(=O)C2CN3CCC2CC3)cc1 ; Cc1cccc(C(=O)C2CN3CCC2CC3)c1C ; O=C(c1nc2ccccc2s1)C1CN2CCC1CC2 ; Cc1ccc(Cl)cc1C(=O)C1CN2CCC1CC2]

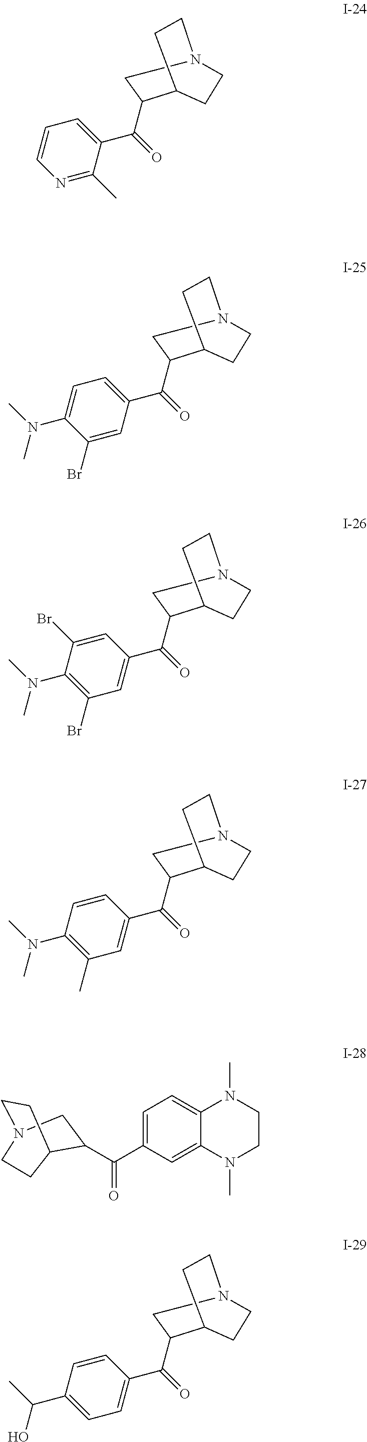 [SMILES: Cc1ncccc1C(=O)C1CN2CCC1CC2 ; CN(C)c1ccc(C(=O)C2CN3CCC2CC3)cc1Br ; CN(C)c1c(Br)cc(C(=O)C2CN3CCC2CC3)cc1Br ; Cc1cc(C(=O)C2CN3CCC2CC3)ccc1N(C)C ; CN1CCN(C)c2cc(C(=O)C3CN4CCC3CC4)ccc21 ; CC(O)c1ccc(C(=O)C2CN3CCC2CC3)cc1]

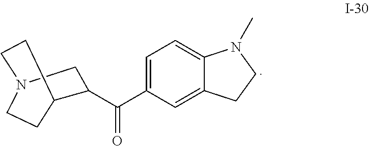 [SMILES: CN1CCc2cc(C(=O)C3CN4CCC3CC4)ccc21]

26. The compound of claim 28, wherein the compound is selected from:

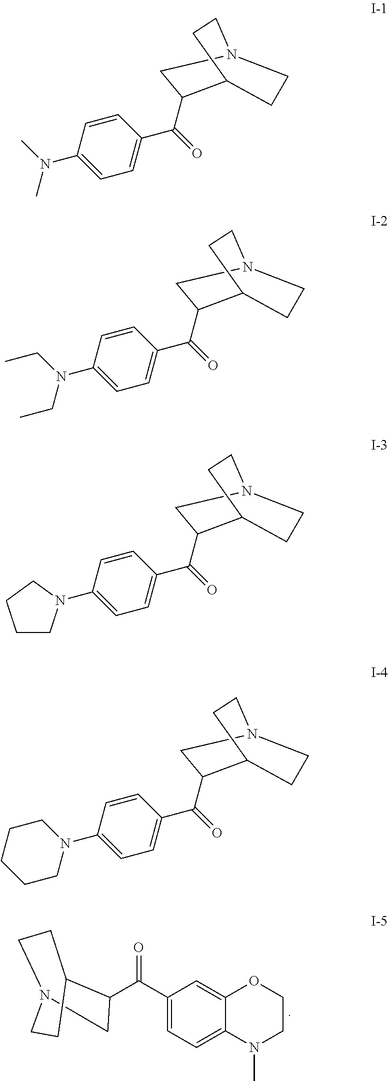 [SMILES: CN(C)c1ccc(C(=O)C2CN3CCC2CC3)cc1 ; CCN(CC)c1ccc(C(=O)C2CN3CCC2CC3)cc1 ; O=C(c1ccc(N2CCCC2)cc1)C1CN2CCC1CC2 ; O=C(c1ccc(N2CCCCC2)cc1)C1CN2CCC1CC2 ; CN1CCOc2cc(C(=O)C3CN4CCC3CC4)ccc21]

27. A compound for inhibiting choline kinase, the compound selected from the following compounds:

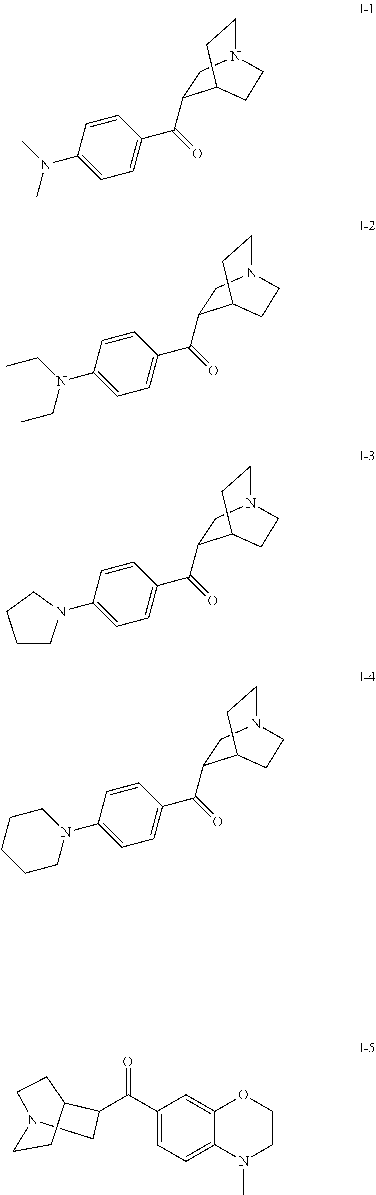 [SMILES: CN(C)c1ccc(C(=O)C2CN3CCC2CC3)cc1 ; CCN(CC)c1ccc(C(=O)C2CN3CCC2CC3)cc1 ; O=C(c1ccc(N2CCCC2)cc1)C1CN2CCC1CC2 ; O=C(c1ccc(N2CCCCC2)cc1)C1CN2CCC1CC2 ; CN1CCOc2cc(C(=O)C3CN4CCC3CC4)ccc21]

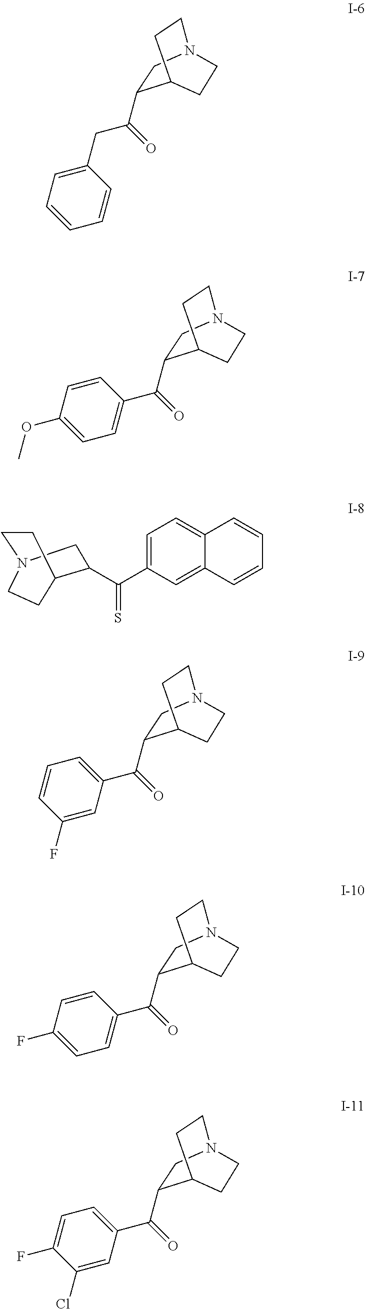 [SMILES: O=C(Cc1ccccc1)C1CN2CCC1CC2 ; COc1ccc(C(=O)C2CN3CCC2CC3)cc1 ; S=C(c1ccc2ccccc2c1)C1CN2CCC1CC2 ; O=C(c1cccc(F)c1)C1CN2CCC1CC2 ; O=C(c1ccc(F)cc1)C1CN2CCC1CC2 ; O=C(c1ccc(F)c(Cl)c1)C1CN2CCC1CC2]

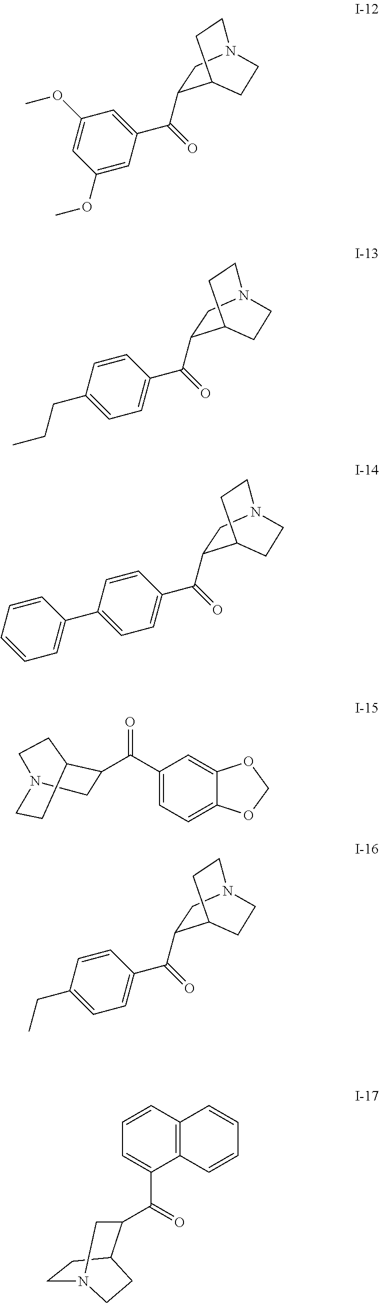 [SMILES: COc1cc(OC)cc(C(=O)C2CN3CCC2CC3)c1 ; CCCc1ccc(C(=O)C2CN3CCC2CC3)cc1 ; O=C(c1ccc(-c2ccccc2)cc1)C1CN2CCC1CC2 ; O=C(c1ccc2c(c1)OCO2)C1CN2CCC1CC2 ; CCc1ccc(C(=O)C2CN3CCC2CC3)cc1 ; O=C(c1cccc2ccccc12)C1CN2CCC1CC2]

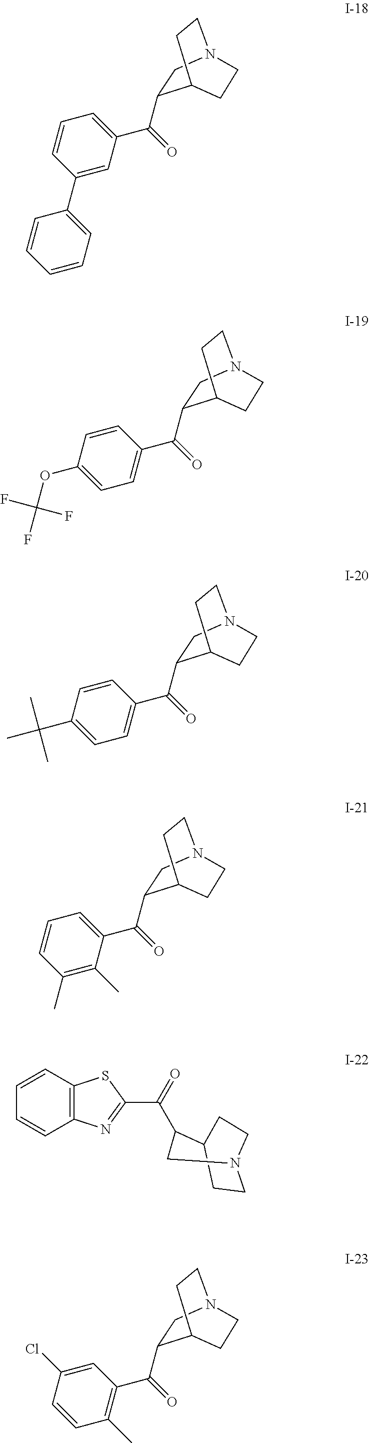 [SMILES: O=C(c1cccc(-c2ccccc2)c1)C1CN2CCC1CC2 ; O=C(c1ccc(OC(F)(F)F)cc1)C1CN2CCC1CC2 ; CC(C)(C)c1ccc(C(=O)C2CN3CCC2CC3)cc1 ; Cc1cccc(C(=O)C2CN3CCC2CC3)c1C ; O=C(c1nc2ccccc2s1)C1CN2CCC1CC2 ; Cc1ccc(Cl)cc1C(=O)C1CN2CCC1CC2]

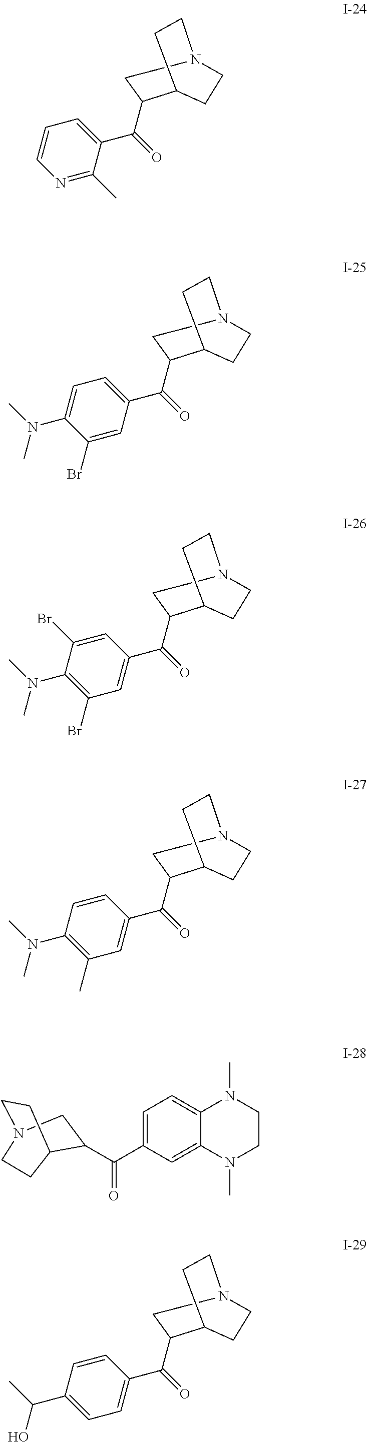 [SMILES: Cc1ncccc1C(=O)C1CN2CCC1CC2 ; CN(C)c1ccc(C(=O)C2CN3CCC2CC3)cc1Br ; CN(C)c1c(Br)cc(C(=O)C2CN3CCC2CC3)cc1Br ; Cc1cc(C(=O)C2CN3CCC2CC3)ccc1N(C)C ; CN1CCN(C)c2cc(C(=O)C3CN4CCC3CC4)ccc21 ; CC(O)c1ccc(C(=O)C2CN3CCC2CC3)cc1]

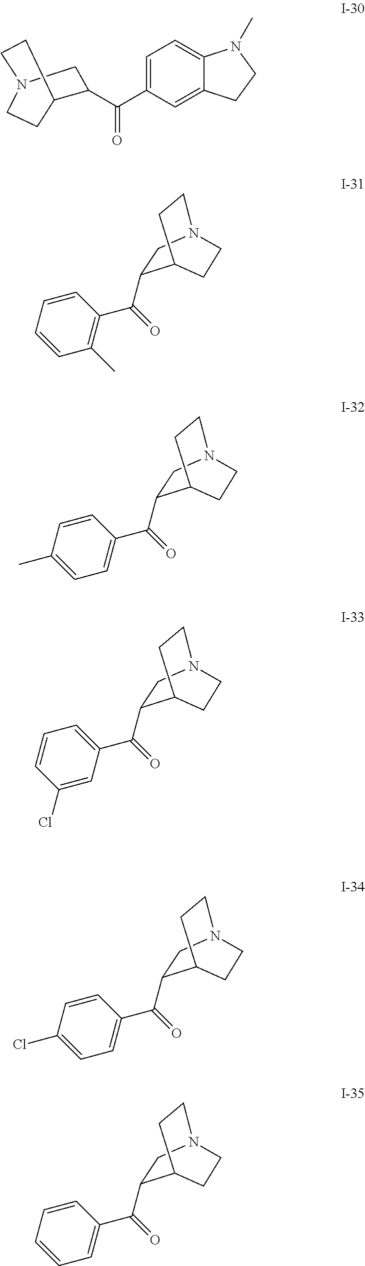 [SMILES: CN1CCc2cc(C(=O)C3CN4CCC3CC4)ccc21 ; Cc1ccccc1C(=O)C1CN2CCC1CC2 ; Cc1ccc(C(=O)C2CN3CCC2CC3)cc1 ; O=C(c1cccc(Cl)c1)C1CN2CCC1CC2 ; O=C(c1ccc(Cl)cc1)C1CN2CCC1CC2 ; O=C(c1ccccc1)C1CN2CCC1CC2]

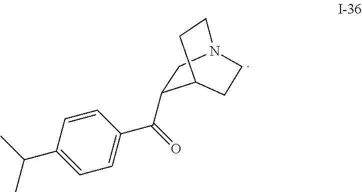 [SMILES: CC(C)c1ccc(C(=O)C2CN3CCC2CC3)cc1]

28. The compound of claim 30 selected from the following compounds:

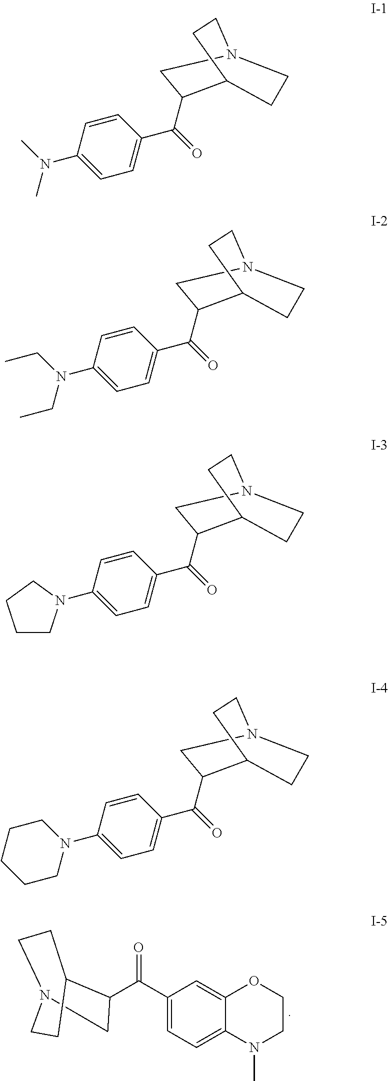 [SMILES: CN(C)c1ccc(C(=O)C2CN3CCC2CC3)cc1 ; CCN(CC)c1ccc(C(=O)C2CN3CCC2CC3)cc1 ; O=C(c1ccc(N2CCCC2)cc1)C1CN2CCC1CC2 ; O=C(c1ccc(N2CCCCC2)cc1)C1CN2CCC1CC2 ; CN1CCOc2cc(C(=O)C3CN4CCC3CC4)ccc21]

29. A composition comprising a compound of any one of claims 1-31, and a pharmaceutically acceptable carrier, adjuvant, or vehicle.

30. A method of inhibiting kinase activity in a patient comprising administering to said patient.
a. a composition of claim 32; or
b. a compound of any one of claims 1-31.

31. A method of inhibiting kinase activity in a biological sample comprising contacting said biologic sample with:
a. a composition of claim 32; or
b. a compound of any one of claims 1-31.

32. The method of any one of claim 33 or 34, wherein said kinase is ChoK.

33. The method of claim 35, wherein said kinase is ChoKα.

34. The method of claim 35, wherein said kinase is ChoKβ.

35. A method of treating or lessening the severity of a disease or condition of a patient selected from cancer, a proliferative disorder, a gastroenterological disorder, a hematological disorder, an endocrinological disorder, a urological disorder, a cardiac disorder, a neurodegenerative disorder, an autoimmune disorder, a respiratory disorder, a metabolic disorder, an inflammatory disorder, an immunologically mediated disorder, a viral disease, infectious disease, or a bone disorder, comprising the step of administering to said patient:
a. a compound of claim 1; or
b. a composition of claim 32.

36. The method according to claim 38 comprising the additional step of administering to said patient an additional therapeutic agent selected from a chemotherapeutic or anti-proliferative agent, an anti-inflammatory agent, an immunomodulatory or immunosuppressive agent, a neurotrophic factor, an agent for treating cardiovascular disease, an agent for treating destructive bone disorders, an anti-viral agent, an agent for treating blood disorders, or an agent for treating immunodeficiency disorders, wherein;
said additional therapeutic agent is appropriate for the disease being treated; and
said additional therapeutic agent is administered together with said composition as a single dosage form or separately from said composition as part of a multiple dosage form.

37. The method of claim 38, wherein said disease is cancer or malaria.

38. A method of treating malaria in a patient wherein the method comprises administering to the patient:
a. a composition of claim 32; or
b. a compound of any one of claims 1-31.

39. A method of treating cancer in a patient wherein the method comprises administering to the patient:
a. a composition of claim 32; or
b. a compound of any one of claims 1-31.

40. The method of claim 32, wherein said cancer is selected from melanoma, myeloma, leukemia, lymphoma, neuroblastoma, or a cancer selected from colon, breast, gastric, ovarian, cervical, lung, central nervous system (CNS), renal, prostate, bladder, or pancreatic.

41. A process for preparing a compound of formula II: 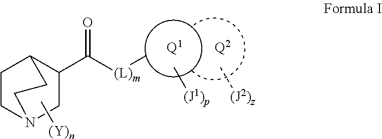
wherein L, m, Y, n, Q¹, Q², J¹, J², z and p are as defined according to any one of claims 1-19, comprising reacting a compound of formula 2-a: 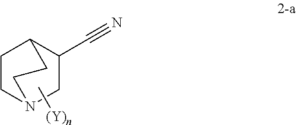
with a compound of formula i, 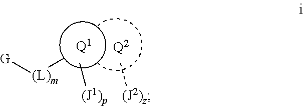
under suitable conditions to generate a nucleophic addition reaction, wherein G is a metal or metal halide.

42. A process for preparing a compound of Formula I: 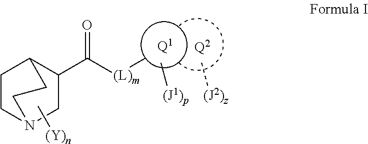
wherein L, m, Y, n, Q¹, Q², J¹, J², z and p are as defined according to any one of claims 1-31, comprising:
a) reacting a compound of formula 3-a: 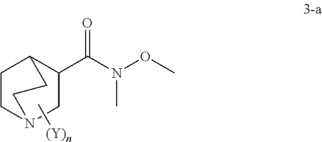
with a compound of formula iii: 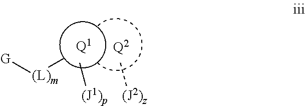
under suitable conditions to produce a displacement reaction, wherein G is lithium or a metal halide;
c) Functionalizing the product of step a) to form a compound of formula I.

43. The process of claim 45, further comprising reacting a compound of formula 3-b: 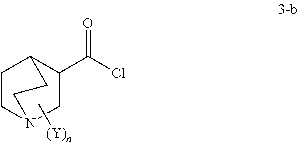
with a compound of formula iv: 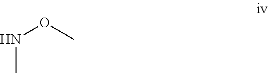
under suitable displacement conditions to form the compound of formula 3a.

44. The process of claim 46, further comprising reacting a compound of formula 3-c: 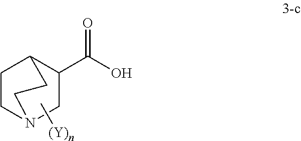
under suitable conditions to produce a nucleophilic addition, whereby a compound of formula 3-b is formed.

45. The process of claim 47, further comprising reacting a compound of formula 3-d: 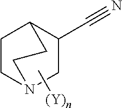
under suitable hydrolysis conditions to form the compound of formula 3-c.
